(19) United States
(12) Patent Application Publication
(10) Pub. No.: US 2026/0253663 A1
(43) Pub. Date: Aug. 27, 2026

(54) DATA DRIVEN SYSTEM AND METHOD TO PREDICT THE IMPACT OF GENE EDITING ON GENE EXPRESSION PROFILES

(71) Applicant: Accenture Global Solutions Limited, Dublin (IE)

(72) Inventors: Maziyar BARAN POUYAN, Petaluma, CA (US); Christophe GUERET, Dublin (IE); Luca COSTABELLO, Newbridge (IE)

(73) Assignee: Accenture Global Solutions Limited, Dublin (IE)

(21) Appl. No.: 19/064,185

(22) Filed: Feb. 26, 2025

(51) Int. Cl. G16B 5/00; G16B 20/00; G16B 40/20

(52) U.S. Cl. CPC G16B 5/00; G16B 20/00; G16B 40/20

(57) ABSTRACT

A method for determining gene to gene interaction based upon gene modification is disclosed. The method includes: (i) extracting a cooperative network implementing an unsupervised regression tree; (ii) classifying a plurality of gene interactions into one of up-regulation category and/or down-regulation category; (iii) creating a gene expression profile matrix based upon gene data and resulting expression weighting functions; (iv) receiving a request to modify a gene and/or an expression of the gene; and (v) simulating an interaction of the modification within the gene expression profile matrix to provide a weighted direction graph.

### TECHNICAL FIELD

The field of the disclosure relates generally to gene editing, and in particular to developing an in-silico model to predict an impact or consequences of gene editing on gene expression profiles.

### REFERENCE TO THE SEQUENCE LISTING

The Sequence Listing submitted 26 Feb. 2025 as an XML file named “D24-209-04871-PR-US 126401-801588—Sequence_Listing”, created on 6 Dec. 2024 and having a size of 20,480 bytes is hereby incorporated by reference pursuant to 37 C.F.R. § 1.52(e)(5).

### BACKGROUND

CRISPR/Cas9 technology has transferred genetic engineering, offering unprecedented precision. In the rapidly evolving field of genetics, therefore, understanding the downstream effects of gene editing is of paramount importance. Currently known efforts for understanding the downstream effects of gene editing are based upon models requiring extensive pre-training. Further, the known techniques are not efficient to allow for rapid adjustments in gene editing process or gene editing strategies, that is crucial from developing precise therapies in medicine and optimizing traits in crops without delays associated with pre-training models gene editing strategies. Accordingly, understanding the complex downstream effects remains a key challenge in biology, affecting both therapeutic and agricultural applications.

This section is intended to introduce the reader to various aspects of art that may be related to various aspects of the present disclosure described or claimed below. This description is believed to be helpful in providing the reader with background information to facilitate a better understanding of the various aspects of the present disclosure. Accordingly, it should be understood that these statements are to be read in this light and not as admissions of prior art.

### SUMMARY

In one aspect, a computer-implemented method for determining gene to gene interaction based upon gene modification is disclosed. The computer-implemented method includes: (i) extracting a cooperative network implementing an unsupervised regression tree; (ii) classifying a plurality of gene interactions into one of up-regulation category and/or down-regulation category; (iii) creating a gene expression profile matrix based upon gene data and resulting expression weighting functions; (iv) receiving a request to modify a gene and/or an expression of the gene; and (v) simulating an interaction of the modification within the gene expression profile matrix to provide a weighted direction graph.

In another aspect, a system of determining gene to gene interaction based upon gene modification is disclosed. The system includes at least one memory configured to store machine executable instructions, and at least one processor communicatively coupled with the at least one memory. The at least one processor is configured to execute the machine executable instructions to perform operations including: (i) extracting a cooperative network implementing an unsupervised regression tree; (ii) classifying a plurality of gene interactions into one of up-regulation category and/or down-regulation category; (iii) creating a gene expression profile matrix based upon gene data and resulting expression weighting functions; (iv) receiving a request to modify a gene and/or an expression of the gene; and (v) simulating an interaction of the modification within the gene expression profile matrix to provide a weighted direction graph.

In yet another aspect, a non-transitory computer-readable medium (CRM) including machine-executable instructions stored thereon is disclosed, The machine-executable instructions, when executed by at least one processor of a computing device, cause the computing device to determine gene to gene interaction based upon gene modification by performing operations including: (i) extracting a cooperative network implementing an unsupervised regression tree; (ii) classifying a plurality of gene interactions into one of up-regulation category and/or down-regulation category; (iii) creating a gene expression profile matrix based upon gene data and resulting expression weighting functions; (iv) receiving a request to modify a gene and/or an expression of the gene; and (v) simulating an interaction of the modification within the gene expression profile matrix to provide a weighted direction graph.

Various refinements exist of the features noted in relation to the above-mentioned aspects. Further features may also be incorporated in the above-mentioned aspects as well. These refinements and additional features may exist individually or in any combination. For instance, various features discussed below in relation to any of the illustrated examples may be incorporated into any of the above-described aspects, alone or in any combination.

### BRIEF DESCRIPTION OF DRAWINGS

The following drawings form part of the present specification and are included to further demonstrate certain aspects of the present disclosure. The disclosure may be better understood by reference to one or more of these drawings in combination with the detailed description of specific examples presented herein.

FIG. 1 is a first example front-end view of a graphical user interface of an editing system.

FIG. 2 is a second example front-end view of the graphical user interface of the editing system.

FIG. 3 is a third example front-end view of the graphical user interface of the editing system.

FIG. 4 is a fourth example front-end view of the graphical user interface of the editing system.

FIG. 5 is a fifth example front-end view of the graphical user interface of the editing system.

FIG. 6 is a sixth example front-end view of the graphical user interface of the editing system.

FIG. 7 is an example flow-chart of method operations of determining gene to gene interaction based upon gene modification, as described herein.

FIG. 8 illustrates an example computing system that can implement various techniques, processes, functions, or methods described herein.

Corresponding reference characters indicate corresponding parts throughout the several views of the drawings. Although specific features of various examples may be shown in some drawings and not in others, this is for convenience only. Any feature of any drawing may be referenced or claimed in combination with any feature of any other drawing.

Some structural or method features may be shown in specific arrangements and/or orderings in the drawings. However, it should be appreciated that such specific arrangements and/or orderings may not be required. Rather, in some examples, such features may be arranged in a different manner and/or order than shown in the illustrative figures. Additionally, the inclusion of a structural or method feature in a particular figure is not meant to imply that such feature is required in all examples, and, in some examples, it may not be included or may be combined with other features.

### DETAILED DESCRIPTION

The following detailed description and examples set forth preferred materials, components, and procedures used in accordance with the present disclosure. This description and these examples, however, are provided by way of illustration only, and nothing therein shall be deemed to be a limitation upon the overall scope of the present disclosure.

One or more of the following terms may be used in the disclosure, and their definition is provided below.

As described herein, in the rapidly evolving field of genetics, understanding the downstream effects of gene editing is of paramount importance. Various examples described in the present disclosure are directed to develop an in-silico model to predict the consequences of gene editing on the gene expression profile. In various examples, computational biology, machine learning algorithms, and experimental data are integrated to provide a holistic overview of how targeted gene modifications can influence the transcriptomic landscape of an organism. The present predictive models streamline gene editing endeavors by anticipating potential pitfalls, and allow for more precise and tailored therapeutic applications in personalized medicine.

In an aspect, “gene expression” refers to the biosynthesis or production of a gene product, including the transcription and/or translation of the gene product. In an aspect, expression refers to the biosynthesis of a gene product, preferably to the transcription and/or translation of a nucleotide sequence, for example an endogenous gene or a heterologous gene, in a cell. For example, in the case of a structural gene, expression involves transcription of the structural gene into mRNA and—optionally—the subsequent translation of mRNA into one or more polypeptides. In an aspect, expression can refer only to the transcription of the DNA harboring an RNA molecule.

For example, gene YAR015W, known as ADE1 in Saccharomyces cerevisiae (see Saccharomyces Genome Database No. S000000070), is a verified Open Reading Frame on chromosome I, which encodes N-succinyl-5-aminoimidazole-4-carboxamide ribotide (SAICAR) synthetase that is essential for purine nucleotide synthesis. ADE1 is notable because yeast cells lacking adenine that lack this gene accumulate a red pigment and its protein levels rise in response to deoxyribonucleic acid (DNA) replication stress. Alterations in ADE1 can affect adenine biosynthesis and broader metabolic pathways. Using the presently disclosed subject matter, an in-silico modification of the gene can predict how other genes may be impacted.

In some aspects, an editing system (referenced herein as a GeneEditInsight tool) is designed to predict the impact of gene editing on gene expression profiles. By leveraging CRISPR/Cas9 technology, insights into how specific gene edits affect overall gene expression are obtained. The obtained insights enable better decision-making in research and applications ranging from medical therapies to agricultural improvements.

Currently known technologies, however, fall short in various aspects. For example, one artificial intelligence (AI) based model that predicts cellular responses to perturbations using single-cell ribonucleic acid (RNA) sequencing data and is not a data driven or unsupervised approach. Further, the one AI based model does not support extracting single cell gene interaction network (GIN) and does not support intuitive user interface (UI). Similarly, another technology is a framework for learning the response of individual cells to a given perturbation by mapping these unpaired distributions. In the other technology framework that is a deep neural network (DNN) approach, neural optimal transport is leveraged for predicting single-cell perturbation responses. Also, the other technology framework is not data driven or based upon an unsupervised approach. Further, prior technology cannot use the dataset for different organisms. In other words, the dataset trained on yeast cannot be used for animals.

The presently disclosed editing system approach supports data driven or unsupervised approach, machine learning and artificial intelligence-based techniques, and includes an intuitive UI. Further, the disclosed editing system also extracts data driven GIN from input data and extract co-expressed cluster genes from GIN. The editing system, which does not have a pre-training requirement allows for real-time adaptability and features an interactive UI making it accessible for immediate and user-friendly analysis of gene interactions. Additionally, by combining a data-driven approach with an interactive UI, the editing system offers precise predictions while enabling users to actively manipulate and visualize gene editing outcomes, enhancing both accuracy and user experience in precision medicine and agriculture.

The disclosed editing system is an in-silico approach and used for determining an impact of a gene editing process on a gene expression pattern. The example computational approach includes a methodology commences with an input of gene expression profile. The gene expression profile that is used as an input is sourced from either bulk or single-cell data.

In the disclosed methodology of the editing system, using an unsupervised regression tree approach, a first phase of analysis is performed. The unsupervised regression tree approach is based on a Genie3 algorithm. The Genie3 algorithm extracts the cooperative network extraction (CRN) based upon a gene expression data matrix. The editing system leverages the Genie3 algorithm and UpDownReg Network Extraction to generate a Weighted Directed Graph (GIN), modeling gene interactions in real-time from existing data, which allows for immediate insights into gene effects, enhancing utility in dynamic research settings. The editing system then uses an Ordinary Differential Equation (ODE) system in its DynamicGIN phase to simulate the impact of gene edits, providing precise predictions and enabling rapid, tailored interventions in therapeutic and agricultural applications to ensure efficient and accurate gene editing strategy adjustments based on real-time insights, without the need for pre-training.

In other words, the extracted CRN is an application of a novel machine learning technique that is designed to discern the nature of gene interactions, classifying them into up-regulation or down-regulation categories (e.g., UpDownReg Network extraction). A combination of these two components (e.g., up-regulation and down-regulation) gives rise to a weighted directed graph, wherein each node symbolizes a distinct gene (GIN).

Additionally, the effects of either increasing or decreasing the expression of specific genes (nodes) in the network are modeled using an Ordinary Differential Equation (ODE) system, which mathematically captures the ongoing dynamics between gene interactions. By adjusting the expression values of these nodes, the system offers predictions on the impact of gene editing on the larger network, giving foresight into changes across the intricate gene expression web, which also highlight the immediate outcomes of gene editing but also delivers essential insights into the ripple effects that occur within the comprehensive genetic structure (DynamicGIN).

In some aspects, for cooperative interaction network extraction (CRN), each gene is treated as a target variable and the remaining genes as potential predictors. For each target gene, an ensemble of regression trees, such as random forests, is employed to rank other genes based on their importance in predicting the target gene's expression. The strength of the association, as determined by its respective importance score, indicates the likelihood of regulatory relationships. The importance score is derived from gene bulk expression data based upon presence of different gene products under various context, as observed or identified. By systematically evaluating every gene in this manner, a comprehensive network is constructed. The comprehensive network captures the intricate relationships among genes, allowing for the identification of potential regulatory interactions within a given genomic dataset. By way of an example, an importance score of a gene is computed as a sum of a decrease in node impurity caused by the gene across all decision trees in which it appears as a splitting variable. As a result, a directed graph is created, with nodes denoting genes and edges denoting regulatory interactions, for example, a gene A controls a gene B, according to an edge from a node A associated with the gene A to a node B associated with the gene B.

In some aspects, in the UpDownReg Network Extraction operation, for each gene i, a Random Forest regression model is generated in which the gene i is the target gene and all other genes are predictor genes. For each predictor gene j in the Random Forest regression model for target gene i, two subsets of the data are randomly sampled. In one of the two subsets, an expression of gene j is higher than its median value, and in another of the two subsets, the expression gene j is lower than that its median value. The values of the target gene i are predicted, using the trained Random Forest regression model, based upon the two subsets described herein. Accordingly, if the predicted value for the higher-expression subset is greater than the lower-expression subset, a positive interaction is inferred; otherwise, a negative interaction is inferred.

The combination of the CRN network and the UpDownReg Network provides or generates a weighted directed graph in which each node represents a distinct gene (GIN). In the GIN graph, each node represents a gene, and each link between two genes indicates a potential direct interaction. For instance, gene A influences the expression of gene B, with a link weight of 0.6 indicating the strength of this interaction. Interaction values range from 0 to 1, where 0 signifies a low interaction and 1 signifies a high interaction. Positive values represent an upregulation effect on target genes, while negative values indicate a downregulation impact.

In some aspects, with regards to the comprehensive genetic structure (DynamicGIN), an ordinary differential equation (ODE) system is formulated to model temporal changes in gene expression levels based on the regulatory effects captured in the adjacency matrix of calculated GIN. The ODE system considers both the regulatory influences of other genes and a decay term for each gene's expression. With an initial condition, the ODE system is solved over a specified time interval, producing a time-course representation of all genes' expression levels. By way of an example, for a network of n genes, the state of the system at time t is represented by a vector X (t) that is [X₁(t), X₂(t), . . . , Xₙ(t)]^{T}, where Xᵢ(t) denotes the expression level of gene i at time t and the adjacency matrix A represents the regulatory influences among the genes (adjacency of GIN), where an element aᵢⱼ represents the regulatory effect of gene j on gene i. The ODE system is thus formulated as

$$\frac{ⅆ x}{ⅆ t} = A . X - δ \cdot X ,$$

wherein

$$\frac{ⅆ x}{ⅆ t}$$

is the rate of change of expression levels, A.X represents the regulatory effects of all genes on each other, and δ·X represents the decay term for each gene's expression, with δ being a vector of the same dimensions as X, containing the decay rates for each gene. The ODEs are solved numerically over a defined time range, with the initial condition, and the results are then visualized to show the change in expression levels over time for all genes.

In some aspects, operations may include uploading a comma separated value (CSV) file including gene expression data as an input file. The gene expression data may be presented as rows*samples. The input file is processed and loaded into the particular editing system, for example, the GeneEditInsight program. Upon loading of the data from the input file, a dynamic two-dimensional (2D) Uniform Manifold Approximation and Projection (UMAP) displaying many different types of data. In the present case, the 2D UMAP presents visualization of gene expression profiles in a graphical user interface. A particular gene of interest may be selected, for example, by using a “Select Target Gene” feature of the graphical user interface for tracking its expression variations across the dataset (e.g., the gene expression data). Additionally, a particular sample may be selected or chosen as a starting point for an expression state for further refining the analysis. By way of an example, the starting point for the expression state may be selected using a “Select Current Gene Expression” option of the graphical user interface. Alternatively, or additionally, the starting point for the expression state may be selected by directly clicking on a data point in the displayed visualization on the graphical user interface. Upon selecting the starting point and selecting “Run Analysis” option of the graphical user interface, one or more machine learning algorithms may be executed for simulating and predicting changes in the gene expression profile.

Upon completion of the analysis, different tabs may be selected to explore varied analytical perspectives. By way of an example, the ‘Dynamic Gene Expression Analysis’ tab may be selected for displaying one or more insightful plots illustrating the temporal expression patterns of sampled genes. Additionally, a detailed table may also be displayed presenting key information such as, including but not limited to, the predicted status of each gene. The predicted status of each gene may be either an upregulated or downregulated over time. Further, for comprehensive analysis, the complete list of these dynamic expression changes may be saved, and, thereby, ensuring availability of all the data for in-depth examination and for any future references.

In some aspects, complexities of gene interactions may be viewed or unveiled using a “Gene Regulatory Network” tab of the graphical user interface in which genes are organized in discernible clusters showcased within a graphical context. Distinctive colors may be used to represent unique clusters for a clear visual distinction. A user may interact with the GIN by selecting a gene. Upon selection of the gene by the user on the graphical user interface, a cluster specific to the selected gene and interactions of the selected gene with other genes may be illuminated or displayed as visually distinguishable from other clusters. The visually distinguishable illustration is intuitive and further simplifies the exploration of intricate regulatory relationships, and, thereby, provides a comprehensive understanding of gene connectivity.

In some aspects, the graphical user interface may also display a tab for a focused “Differential Gene Expression Analysis” selection in which genes that exhibit differential expression between two distinct states may be displayed in detail. Two distinct states may include when the target gene is active (or on) and when the target gene is inactive (or off). Accordingly, the “Differential Gene Expression Analysis” tab provides a comparative view, highlighting the genes whose expression levels significantly change in response to the target gene's activity. The “Differential Gene Expression Analysis” is pivotal for understanding the influence of the target gene on the cellular environment and expression of rest of the genes.

In some aspects, the graphical user interface may also include and display a “Pathway Analysis” tab. Upon selecting the “Pathway Analysis” tab, different pathways affecting the selected gene's activity may be displayed. In other words, pathways that become more or less active when the gene is on or off are displayed illustrating the selected gene's impact. Further, the graphical user interface may also display a button providing affordance to the user to export analyzed the analyzed data for further use or reporting.

Accordingly, various aspects as described herein, predicts possible issues or complications as a result of gene editing without a need of a pre-trained machine learning model. As described herein, the present approach focuses on the genomic stage, and in particular transcription, employing a data-driven method to predict the outcomes of gene editing on gene expression profiles. Further, various aspects are directed to understand how changes at the genomic level influence gene expression, offering a different perspective from the epigenetic focus of previous studies. Additionally, various aspects described herein are distinct as they predict the expression profile of a given cell type based on its characteristics. The disclosed method, according to various aspects described herein, involves using cell type as input to forecast gene expression and is fundamentally different from predicting the effects of gene editing on gene expression profiles. Accordingly, the competitive advantage of the disclosed editing system according to various aspects lies in its direct utilization of existing experimental data for predicting gene editing outcomes, enabling immediate analysis of gene interaction effects.

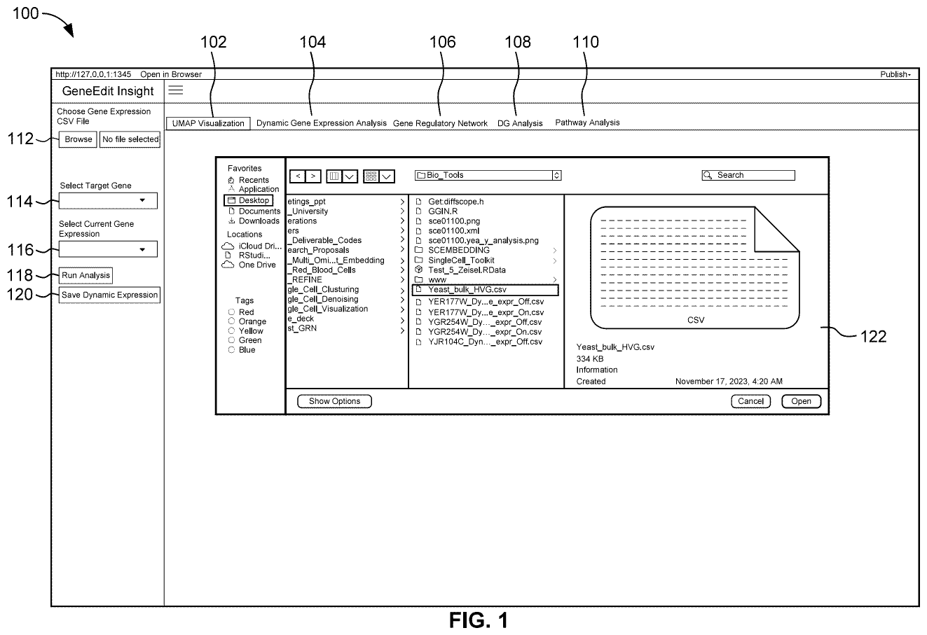

Various aspects are described in detail below with reference to FIG. 1 through FIG. 8.

FIG. 1 is an example front-end view 100 of a graphical user interface of the present editing system demarked as “GeneEditInsight” tool in the example user interfaces (UIs) illustrated herein. As shown in the front-end view 100, a user may upload a comma separated value (CSV) file including gene expression data as an input file by providing a file name in an input field 112. In an example, the user may select the input file by bringing a cursor in the field 112. Upon bringing the cursor in the input field 112, an overlay 122 may be displayed for the user to browse through various directories or folders to select the file name in the input field 112. The selected file may be a CSV file including the gene expression data as rows*samples. The selected input file is processed and loaded into the particular GeneEditInsight tool.

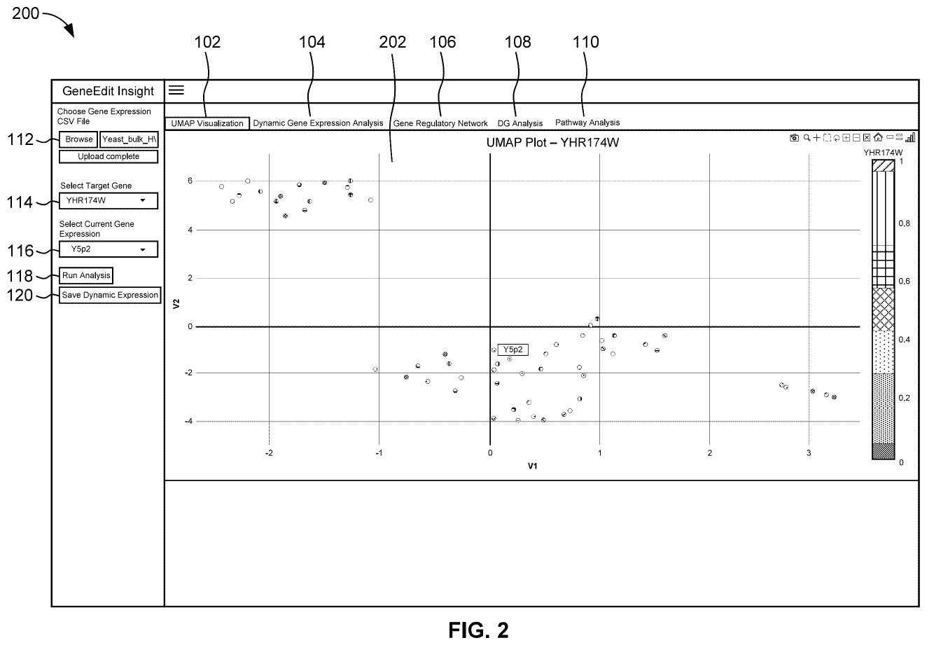

FIG. 2 is an example front-end view 200 of the graphical user interface of the GeneEditInsight tool. As shown in the front-end view 200, upon selecting the csv input file for processing, a two-dimensional (2D) Uniform Manifold Approximation and Projection (UMAP) 202 is displayed when the user selects a tab labeled UMAP visualization 102 in a viewing pane (not labeled in FIG. 1 or FIG. 2). The UMAP visualization 202 displays many different types of data. In the present case, the 2D UMAP 202 presents visualization of gene expression profiles in a graphical user interface. A particular gene of interest may be selected, for example, by using a “Select Target Gene” feature 114 of the graphical user interface for tracking its expression variations across the dataset (e.g, the gene expression data). By way of an example, the “Select Target Gene” feature 114 may be provided as a pull-down menu, and the user may select the gene using a pull-down option. Alternatively, the user may provide a partial name of the target gene to narrow down the genes being displayed for the user to select from using the pull-down option.

Additionally, a particular sample may be selected or chosen as a starting point for an expression state for further refining the analysis. By way of an example, the starting point for the expression state may be selected using a “Select Current Gene Expression” option 116 of the graphical user interface. The “Select Current Gene Expression” option 116 may be implemented similar to the “Select Target Gene” feature 114. Alternatively, or additionally, the starting point for the expression state may be selected by directly clicking on a data point in the displayed UMAP visualization 202 on the graphical user interface. Upon selecting the starting point and selecting or clicking “Run Analysis” option 118 of the graphical user interface, one or more machine learning algorithms may be executed for simulating and predicting changes in the gene expression profile. The “Save Dynamic Expression” option 120 of the graphical user interface allows the user to save the performed dynamic expression in a local memory or database.

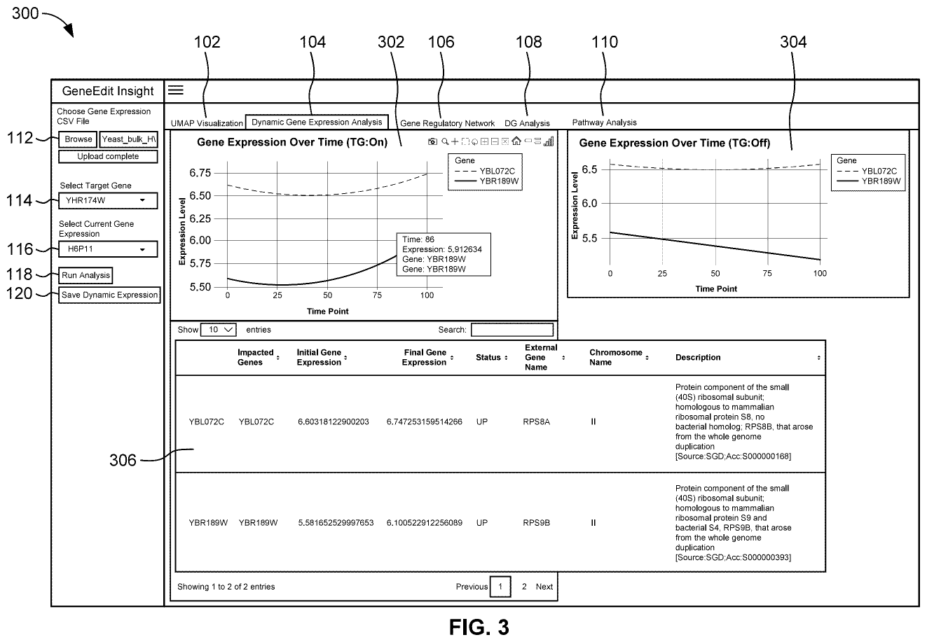

Upon completion of the analysis, different tabs may be selected to explore varied analytical perspectives as described herein with reference to FIG. 3 through FIG. 6. FIG. 3 is an example front-end view 300 of the graphical user interface of the GeneEditInsight tool displayed when the user selects the ‘Dynamic Gene Expression Analysis’ tab 104 for displaying one or more insightful plots 302 and 304 illustrating the temporal expression patterns of sampled genes. Additionally, a detailed table 306 may also be displayed presenting key information such as, including but not limited to, the predicted status of each gene. The predicted status of each gene may be either an upregulated or downregulated over time. Further, for comprehensive analysis, the complete list of these dynamic expression changes may be saved, and, thereby, availability of all the data for in-depth examination and for any future references is ensured.

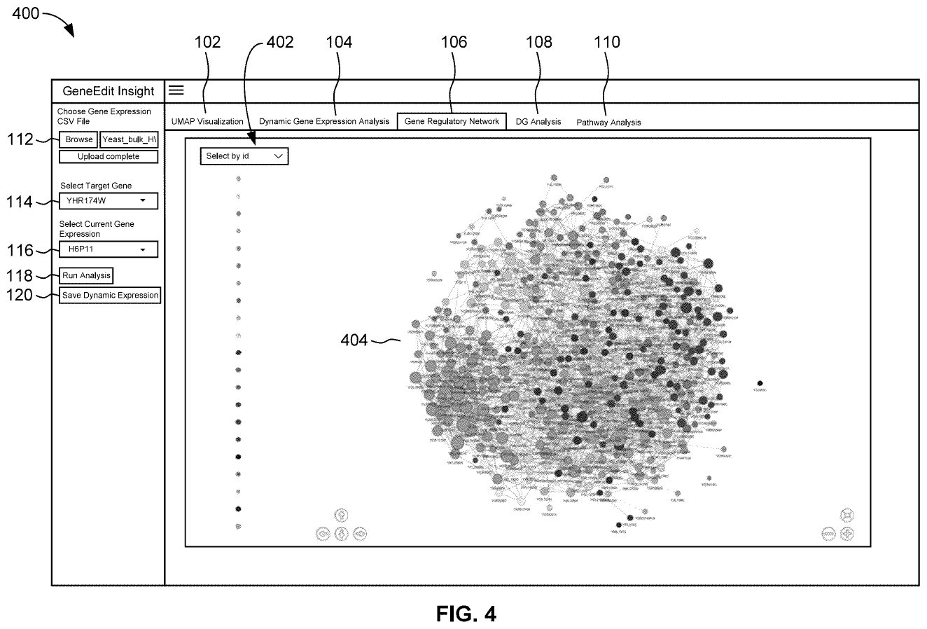

FIG. 4 is an example front-end view 400 of the graphical user interface of the GeneEditInsight tool displayed when the user selects the “Gene Regulatory Network” tab 106. Upon the user selecting the “Gene Regulatory Network” tab 106, complexities of gene interactions are displayed on the graphical user interface as organized in discernible clusters. Distinctive colors may be used to represent unique clusters for a clear visual distinction. A user may interact with the GIN by selecting a gene, for example, using an input field 402. The user may enter text input in the input field 402 for a quick lookup of the gene, or use the pull-down menu to select the gene.

Upon selection of the gene by the user on the graphical user interface, a cluster specific to the selected gene and interactions of the selected gene with other genes may be illuminated or displayed as visually distinguishable from other clusters as shown in FIG. 4 as 404. The visually distinguishable illustration is intuitive and further simplifies the exploration of intricate regulatory relationships, and, thereby, provides a comprehensive understanding of gene connectivity.

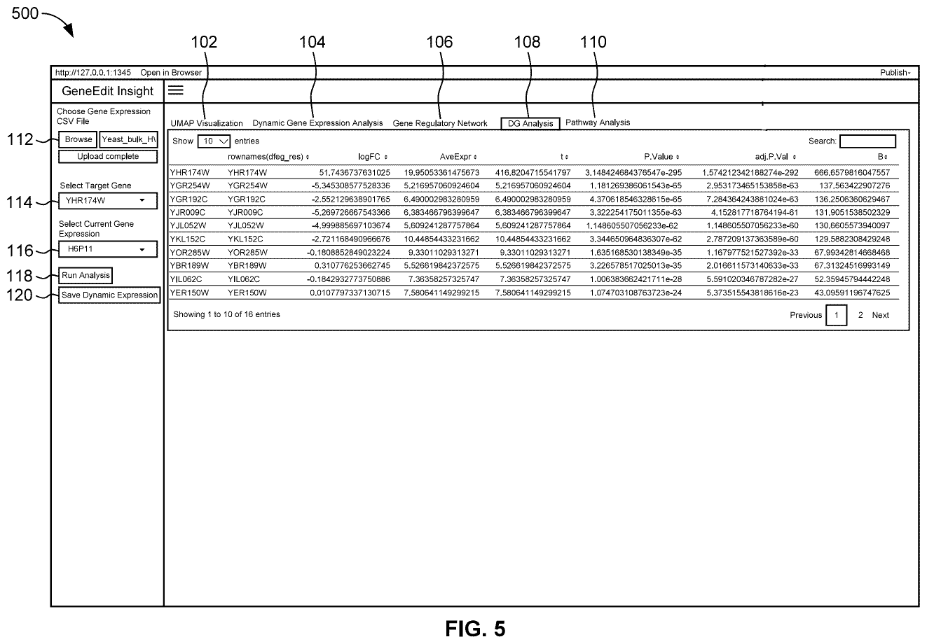

FIG. 5 is an example front-end view 500 of the graphical user interface of the GeneEditInsight tool displayed when the user selects the “Differential Gene Expression Analysis” tab 108. Upon selecting the “Differential Gene Expression Analysis” tab 108 by the user, genes that exhibit differential expression between two distinct states may be displayed in detail. As shown in FIG. 5, the first two columns of the table (not numbered) display r the genes being compared, and the other columns display information about differential gene expression information. In other words, the table shown in FIG. 5 illustrates comparison of expression of two genes in the first two columns, and the expression of difference in the next columns. The expression of difference is related to the quantity of gene product produced, and the statistical significance of the expression of difference (p value). Two distinct states may include when the target gene is active (or on) and when the target gene is inactive (or off). Accordingly, the “Differential Gene Expression Analysis” tab 108 provides a comparative view, highlighting the genes whose expression levels significantly change in response to the target gene's activity. The “Differential Gene Expression Analysis” 108 provides or displays pivotal information for understanding the influence of the target gene on the cellular environment and expression of rest of the genes.

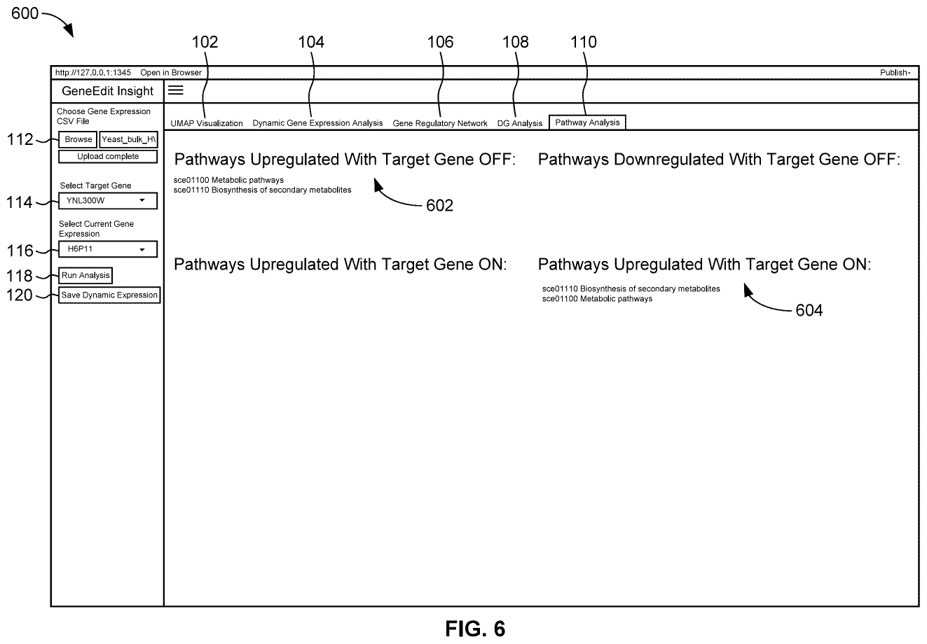

FIG. 6 is an example front-end view 600 of the graphical user interface of the GeneEditInsight tool displayed when the user selects a “Pathway Analysis” tab 110. Upon selecting the “Pathway Analysis” tab 110, different pathways, for example, 602 and 604, affecting the selected gene's activity are displayed. In other words, pathways that become more or less active when the gene is on or off are displayed illustrating the selected gene's impact. Further, the graphical user interface may also display a button providing affordance to the user to export analyzed the analyzed data for further use or reporting.

Accordingly, various aspects as described herein, predicts possible issues or complications as a result of gene editing without a need of a pre-trained machine learning model. While a breakthrough in epigenome editing technology utilizes a modular CRISPR-based system to precisely program epigenetic modifications, which allows researchers to dissect the causal relationships between chromatin marks and their biological effects for providing new insights into gene regulation at the epigenetic level; however, this approach is based upon a supervised machine learning method. In contrast, various aspects as described herein, focus on the genomic stage, and in particular transcription, employing a data-driven method to predict the outcomes of gene editing on gene expression profiles. Further, various aspects are directed to understand how changes at the genomic level influence gene expression, offering a different perspective from the epigenetic focus of previous studies. Additionally, various aspects described herein are distinct as they predict the expression profile of a given cell type based on its characteristics. The disclosed method, according to various aspects described herein, involves using cell type as input to forecast gene expression, and it is fundamentally different from predicting the effects of gene editing on gene expression profiles. Accordingly, the competitive advantage of the disclosed editing system, according to various aspects, lies in its direct utilization of existing experimental data for predicting gene editing outcomes, enabling immediate analysis of gene interaction effects.

In an aspect, genome-wide association studies (GWAS) have been increasingly successful at identifying single-nucleotide polymorphisms (SNPs) with statistically significant association to a variety of diseases and gene sets significantly enriched for SNPs with moderate association. Genetic interactions generally refer to a combination of two or more genes whose contribution to a phenotype cannot be completely explained by their independent effects. One example of an extreme genetic interaction is synthetic lethality where two mutations, neither of which is lethal on its own, combine to generate a lethal double mutant phenotype. Thus, genetic interactions may explain how relatively benign variation can combine to generate more extreme phenotypes, including complex human diseases.

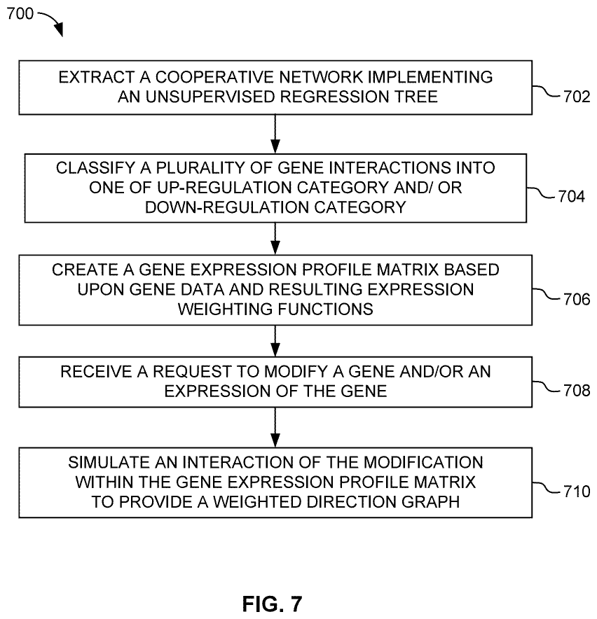

FIG. 7 is an example flow-chart 700 of method operations of determining gene to gene interaction based upon gene modification, as described herein. The method operations include extracting 702 a cooperative network implementing an unsupervised regression tree. As described herein, the purpose of the unsupervised regression tree is to identify patterns or structure in data without using labeled examples. By way of an example, clustering is a common unsupervised learning technique that groups similar examples together based on their features, and unsupervised tree models are an adaptive way of generating clusters of samples. The unsupervised regression trees include unsupervised random forests and decision trees for axis unimodal clustering.

The method operations include classifying 704 a plurality of gene interactions into one of an up-regulation category and/or a down-regulation category. An up-regulation category of the gene interactions refers to a graphical representation of how different genes within a specific category are interacting with each other, where the focus is primarily on genes that are being “up-regulated”—meaning their expression levels are significantly increased under certain conditions, for example, leading to enhanced protein production. Similarly, a down-regulation category of the gene interactions refers to a visual representation of how different genes interact with each other within a biological system, where the focus is specifically on genes that are being “down-regulated”—meaning their expression levels are being decreased, often in response to a specific stimulus or condition. In other words, it shows which genes are interacting with each other while their activity is being reduced. By way of an example, classifying the plurality of gene interactions into one of the up-regulation category and/or the down-regulation category is based upon one or more predictor genes in the model for a target gene.

The method operations include creating 706 a gene expression profile matrix based upon gene data and resulting expression weighting functions. The gene expression profile matrix is a table of data that represents the expression of genes in a cell or tissue, in which rows represents genes, columns represent specific conditions of the array measurement, and entries of the gene expression profile matrix Represent the number of reads (expression level) of a particular gene in a given sample. As described herein, gene expression profiling is the process of measuring the activity of thousands of genes at once to create a picture of global gene expression in a cell population.

The method operations include receiving 708 a request to modify a gene and/or an expression of the gene and simulating 710 an interaction of the modification within the gene expression profile matrix to provide a weighted direction graph. The method operations include generating a random sample of a first subset in which an expression of a predictor gene is higher than a median expression and generating a random sample of a second subset in which an expression of the predictor gene is lower than the median expression. Further, the method operations include predicting a first value for the first subset and predicting a second value for the second subset using the unsupervised regression trees; inferring a positive interaction when the first value is higher than the second value; and inferring a negative interaction when the second value is higher the first value. Additionally, the method operations include repeating, for each target gene and predictor gene combination, the generating of the random sample of the first subset, the generating of the random sample of the second subset, predicting the first value, predicting the second value, and inferring one of a positive or negative interaction. The method operations also include determining, based on real-time existing data, a rate of change of expression levels corresponding to the positive or negative interaction of all of the target genes and predictor genes on one another; suggesting a gene editing strategy based upon the rate of change of expression levels.

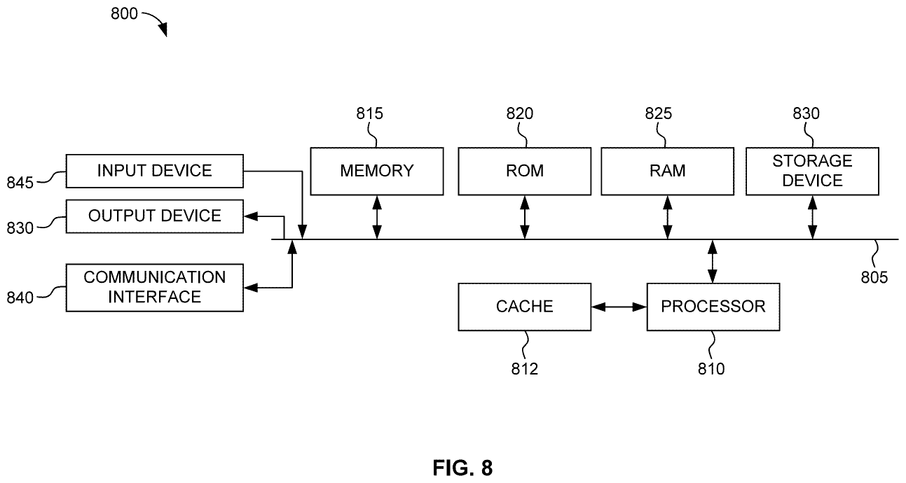

FIG. 8 illustrates an example computing system 800 that can implement various techniques, processes, functions, or methods described herein. The components of computing system 800 are shown in electrical communication with each other using a connection 805, such as a bus. The example computing system 800 includes a processing unit (CPU or processor) 810 and a computing device connection 805 that couples various computing device components, including computing device memory 815, such as a read only memory (ROM) 820 and a random-access memory (RAM) 825, to processor 810.

Computing system 800 can include a cache 812 of high-speed memory connected directly with, in close proximity to, or integrated as part of processor 810. Computing system 800 can copy data from memory 815 and/or storage device 830 to cache 812 for quick access by processor 810. In this way, cache 812 can provide a performance boost that avoids processor 810 delays while waiting for data. These and other modules can control or be configured to control processor 810 to perform various actions. Other computing device memory 815 may be available for use as well. Memory 815 can include multiple different types of memory with different performance characteristics. Processor 810 can include any general-purpose processor, central processing unit (CPU), or graphics processing unit (GPU) in combination with a hardware or software provision configured to control processor 810 and stored in storage device 830, as well as any special-purpose processor where software instructions are incorporated into the processor design. Processor 810 may be a self-contained system, containing multiple cores or processors, a bus, memory controller, cache, etc. A multi-core processor may be symmetric or asymmetric.

Storage device 830 is a non-volatile memory and can be one or more of a hard disk or other types of computer readable media that can store data that are accessible by a computer, such as a magnetic cassette, flash memory card, solid state memory device, digital versatile disk, cartridge, RAM 825, ROM 820, or hybrids thereof. Memory 815 or storage device 830 can include software, code, firmware, etc., for controlling processor 810. Other hardware or software modules are contemplated. Memory 815 and storage device 830 are connected to computing device connection 805. In one aspect, a hardware module that performs a particular function can include the software component stored in a computer-readable medium in connection with the necessary hardware components, such as processor 810, computing device connection 805, and so forth, to carry out the function. In some examples, processor 810 may be programmed by encoding an operation or function using one or more executable instructions and providing the executable instructions in memory 815 or storage device 830.

The processor 810 may be communicatively coupled with a communication interface 840 to communicate with external entities. The communication interface 840 may include one or more of a radio interface, and/or a local area network interface. The processor 810 may be communicatively coupled with an input device 845. The input device 845 may include a keyboard, a mouse, a stylus, or a pen.

In operation, a computer executes computer-executable instructions embodied in one or more computer-executable components stored on one or more computer-readable media to implement aspects of the disclosure described or illustrated herein. The order of execution or performance of the operations in examples of the disclosure illustrated and described herein is not essential, unless otherwise specified. That is, the operations may be performed in any order, unless otherwise specified, and disclosed examples may include additional or fewer operations than those disclosed herein. For example, it is contemplated that executing or performing a particular operation before, contemporaneously with, or after another operation is within the scope of aspects of the disclosure.

In some examples, the system may be configured to implement machine learning models, including neural network, that “learns” to analyze, organize, process, and/or validate data without being explicitly programmed. Machine learning may be implemented through machine learning (ML) methods and algorithms. In some examples, a machine learning (ML) module may be configured to implement ML methods and algorithms. In some examples, ML methods and algorithms may be applied to data inputs and generate machine learning (ML) outputs. Data inputs may include but are not limited to: analog and digital signals, sensor data, image data, video data, datasets stored in one or more databases, and the like. ML outputs may include but are not limited to: digital signals, matrices, predictions and guidance, and the like. In some examples, data inputs may include certain ML outputs.

In some examples, at least one of a plurality of ML methods and algorithms may be applied, which may include but are not limited to: linear or logistic regression, instance-based algorithms, regularization algorithms, decision trees, Bayesian networks, cluster analysis, association rule learning, artificial neural networks, deep learning, recurrent neural networks, Monte Carlo search trees, generative adversarial networks, dimensionality reduction, and support vector machines. In various examples, the implemented ML methods and algorithms may be directed toward at least one of a plurality of categorizations of machine learning, such as supervised learning, unsupervised learning, and reinforcement learning.

In some examples, ML methods and algorithms may be directed toward supervised learning, which involves identifying patterns in existing data to make predictions about subsequently received data. Specifically, ML methods and algorithms directed toward supervised learning are “trained” through training data, which includes example inputs and associated example outputs. Based on the training data, the ML methods and algorithms may generate a predictive function which maps outputs to inputs and utilize the predictive function to generate ML outputs based on data inputs. The example inputs and example outputs of the training data may include any of the data inputs or ML outputs described above. For example, a ML module may receive training data comprising data associated with different patients and their corresponding outcomes, generate a model which maps the patient data to the outcome data, and recognize potential future outcomes for patients.

In some examples, ML methods and algorithms may be directed toward unsupervised learning, which involves finding meaningful relationships in unorganized data. Unlike supervised learning, unsupervised learning does not involve user-initiated training based on example inputs with associated outputs. Rather, in unsupervised learning, unlabeled data, which may be any combination of data inputs and/or ML outputs as described above, is organized according to an algorithm-determined relationship. In some examples, a ML module coupled to or in communication with the design system or integrated as a component of the design system receives unlabeled data, and the ML module employs an unsupervised learning method such as “clustering” to identify patterns and organize the unlabeled data into meaningful groups. The newly organized data may be used, for example, to extract further information about the potential classifications.

In some examples, ML methods and algorithms may be directed toward reinforcement learning, which involves optimizing outputs based on feedback from a reward signal. Specifically, ML methods and algorithms directed toward reinforcement learning may receive a user-defined reward signal definition, receive a data input, utilize a decision-making model to generate a ML output based on the data input, receive a reward signal based on the reward signal definition and the ML output, and alter the decision-making model so as to receive a stronger reward signal for subsequently generated ML outputs. The reward signal definition may be based on any of the data inputs or ML outputs described above. In some examples, a ML module implements reinforcement learning in a user recommendation application. The ML module may utilize a decision-making model to generate a ranked list of options based on user information received from the user and may further receive selection data based on a user selection of one of the ranked options. A reward signal may be generated based on comparing the selection data to the ranking of the selected option. The ML module may update the decision-making model such that subsequently generated rankings more accurately predict optimal constraints.

Some disclosed examples involve the use of one or more electronic processing or computing devices. In an aspect, the terms “processor” and “computer” and related terms, e.g., “processing device,” and “computing device” are not limited to just those integrated circuits referred to in the art as a computer, but broadly refers to a processor, a processing device or system, a general purpose central processing unit (CPU), a graphics processing unit (GPU), a microcontroller, a microcomputer, a programmable logic controller (PLC), a reduced instruction set computer (RISC) processor, a field programmable gate array (FPGA), a digital signal processor (DSP), an application specific integrated circuit (ASIC), and other programmable circuits or processing devices capable of executing the functions described herein, and these terms are used interchangeably herein. These processing devices are generally “configured” to execute functions by programming or being programmed, or by the provisioning of instructions for execution. The above examples are not intended to limit in any way the definition or meaning of the terms processor, processing device, and related terms.

The various aspects illustrated by logical blocks, modules, circuits, processes, algorithms, and algorithm steps described above may be implemented as electronic hardware, software, or combinations of both. Certain disclosed components, blocks, modules, circuits, and steps are described in terms of their functionality, illustrating the interchangeability of their implementation in electronic hardware or software. The implementation of such functionality varies among different applications given varying system architectures and design constraints. Although such implementations may vary from application to application, they do not constitute a departure from the scope of this disclosure.

Aspects of examples implemented in software may be implemented in program code, application software, application programming interfaces (APIs), firmware, middleware, microcode, hardware description languages (HDLs), or any combination thereof. A code segment or machine-executable instruction may represent a procedure, a function, a subprogram, a program, a routine, a subroutine, a module, a software package, a class, or any combination of instructions, data structures, or program statements. A code segment may be coupled to, or integrated with, another code segment or an electronic hardware by passing or receiving information, data, arguments, parameters, memory contents, or memory locations. Information, arguments, parameters, data, etc. may be passed, forwarded, or transmitted via any suitable means including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the disclosed functions may be embodied, or stored, as one or more instructions or code on or in memory. In the examples described herein, memory includes non-transitory computer-readable media, which may include, but is not limited to, media such as flash memory, a random-access memory (RAM), read-only memory (ROM), erasable programmable read-only memory (EPROM), electrically erasable programmable read-only memory (EEPROM), and non-volatile RAM (NVRAM). In an aspect, the term “non-transitory computer-readable media” is intended to be representative of any tangible, computer-readable media, including, without limitation, non-transitory computer storage devices, including, without limitation, volatile and non-volatile media, and removable and non-removable media such as a firmware, physical and virtual storage, CD-ROM, DVD, and any other digital source such as a network, a server, cloud system, or the Internet, as well as yet to be developed digital means, with the sole exception being a transitory propagating signal. The methods described herein may be embodied as executable instructions, e.g., “software” and “firmware,” in a non-transitory computer-readable medium. In an aspect, the terms “software” and “firmware” are interchangeable and include any computer program stored in memory for execution by personal computers, workstations, clients, and servers. Such instructions, when executed by a processor, configure the processor to perform at least a portion of the disclosed methods.

In an aspect, an element or step recited in the singular and proceeded with the word “a” or “an” should be understood as not excluding plural elements or steps unless such exclusion is explicitly recited. Furthermore, references to “one embodiment” of the disclosure or an “exemplary” or “example” embodiment are not intended to be interpreted as excluding the existence of additional embodiments that also incorporate the recited features. Likewise, limitations associated with “one embodiment” or “an embodiment” should not be interpreted as limiting to all embodiments unless explicitly recited.

Disjunctive language such as the phrase “at least one of X, Y, or Z,” unless specifically stated otherwise, is generally intended, within the context presented, to disclose that an item, term, etc. may be either X, Y, or Z, or any combination thereof (e.g., X, Y, and/or Z). Likewise, conjunctive language such as the phrase “at least one of X, Y, and Z,” unless specifically stated otherwise, is generally intended, within the context presented, to disclose at least one of X, at least one of Y, and at least one of Z.

In an aspect, a plurality can comprise one or more of something such as, for example, gene interactions.

In an aspect, “gene” can refer to a region operably linked to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (downstream) the coding region (open reading frame, ORF). In an aspect, the term “structural gene” is intended to mean a DNA sequence that is transcribed into mRNA, which is then translated into a sequence of amino acids characteristic of a specific polypeptide.

In an aspect, gene to gene interactions can be synergistic or antagonistic. In an aspect, gene interaction is the process by which one gene influences the expression of another gene.

In an aspect, gene interactions are divided into two categories: (i) Allelic or Non-epistatic Gene Interaction (gene interaction occurs between the alleles of a single gene) or (ii) Non-allelic or Epistatic Gene Interaction (gene interaction involves interactions between genes on identical or distinct chromosomes). Gene-to-gene interaction is important for understanding the structure and function of genetic pathways, as well as the evolutionary dynamics of complex genetic systems.

Gene interactions are divided into intracellular gene interactions and intercellular gene interactions. Cells communicate with each other through a variety of signal transduction mechanisms, which are essential interactions between genes. In an aspect, there are two types of gene-to-gene interaction: biological epistasis, which has a biological basis, and statistical epistasis, which describes deviation from additivity in a linear statistical model.

In an aspect, “gene regulatory network” or GRN refers to a gene regulatory network (GRN) is a model of the different type of gene interaction. The main interactions are up-regulation and down-regulation. The GRN of a particular organism is a tool that can be used to predict a knock-on effect when modifying the expression of a particular target gene.

In an aspect, up-regulation (or upregulation) can mean an increase in expression level and/or activity level of one or more genes and/or one or more proteins and/or one or more transgenes. For example, in an aspect, up-regulation can mean increased gene expression and/or increased protein expression and/or increased transgene expression. In an aspect, the level and/or expression of a gene can be up-regulated. In an aspect, the level and/or expression of a protein can be up-regulated. In an aspect, up-regulation of expression and/or activity of one or more genes or one or more proteins or one or more transgenes can be compared to a reference level or a control level. For example, in an aspect, a preRNA, mRNA, rRNA, tRNA, expressed by the target gene and/or of the protein product encoded by it, can be up-regulated.

In an aspect, down-regulation (or downregulation) can mean a decrease in expression level and/or activity level of one or more genes and/or one or more proteins and/or one or more transgenes. For example, in an aspect, down-regulation can mean decreased gene expression and/or decreased protein expression and/or decreased transgene expression. In an aspect, the level and/or expression of a gene can be down-regulated. In an aspect, the level and/or expression of a protein can be down-regulated. In an aspect, down-regulation of expression and/or activity of one or more genes or one or more proteins or one or more transgenes can be compared to a reference level or a control level. For example, in an aspect, a preRNA, mRNA, IRNA, tRNA, expressed by the target gene and/or of the protein product encoded by it, can be down-regulated.

Genes rarely act in isolation; instead, they interact with each other and make up gene regulatory networks to function as a whole. The study of this mechanism is crucial for understanding the properties and functions of genes, which help reveal the genetic architecture of complex traits and diseases. Although genetic experiments can be conducted to discover interactions among genes, this approach can be costly and time consuming. Alternatively, measurements of gene expression levels reveal gene expression patterns in a specific condition and can be exploited to infer gene regulatory networks. Various approaches have been proposed to infer gene regulatory networks using gene expression data, such as relevance networks, Bayesian networks, Gaussian graphical models, and many others.

The gene regulation network is a biochemical network formed by a group of genes, proteins, small molecules and mutual regulation and control effects among the genes, the proteins and the small molecules, and is a basic and important biological network. The biological control theory combining biology and control theory is an important component of the control theory, and the gene regulation network is an important branch of the biological control theory, which can skillfully apply the control, regulation and cooperation in the biological system to a multi-agent system. The Turing reaction-diffusion model is a classical and effective model for studying biological pattern formation, and has been greatly developed in recent decades.

In an aspect, the gene regulation network modeling is mainly based on the regulation relationship in the gene expression data reasoning network, and is expressed as a topological structure, and belongs to the research of reverse engineering by data mining. The construction of the gene regulation network firstly needs to determine a network model, and then selects a proper modeling algorithm according to the model. Classical network models include Boolean networks, associative networks, differential equations, and Bayesian networks.

For example, in an aspect, a Boolean network makes corresponding simplification to the gene state, and uses Boolean function to replace differential and derivative to describe the correlation between genes. The model has the defects of inaccuracy, the fact that the real gene regulation network topological structure cannot be accurately described only by describing and reflecting the interaction between genes by using a fixed logic rule, and the loss of important expression information is inevitably caused when gene data are discretized. Probabilistic Boolean Network (PBN), which is an extension of the conventional Boolean network, simultaneously quantifies the interaction relationship and sensitivity between genes to solve the uncertainty in the model selection process and improve the accuracy of the model.

In an aspect, associating the network is modeling of the association network is mainly realized by the association degree between gene expression data. The similarity between genes is usually calculated by using measures such as mutual information, Pearson correlation coefficient and the like, and if the similarity between the gene pairs is higher than a certain threshold value, the gene pairs are directly connected in the network. Button et al first calculates the degree of association between all pairs of genes using mutual information, and then sets a threshold value for the mutual information. Later, it was found that if the gene pairs have the same or similar regulatory mechanism, the association between the two genes is higher, especially the target genes of the same transcription factor or the genes on the same biological pathway. To reduce the false positive rate of the constructed network structure and obtain a regulation and control network close to a real topology, the influence of other genes is isolated when the association degree between the gene pairs is calculated.

Gene regulatory networks can be characterized using a system of structural equations, with each equation describing the causal effects of cis-eQTL and the regulatory effects of other genes on a given gene. Such a framework makes it feasible to take a genome-wide survey and to directly reveal interactions among genes. Application of structural equations in genetical genomics studies have been previously demonstrated. Two studies are applicable to constructing gene regulatory networks for a small number of genes. However, genetical genomics experiments usually collect whole-genome gene expressions for a very limited number of samples, therefore the number of genes is much larger than the sample size. For such consideration, another study proposed to apply the adaptive lasso to construct a sparse gene regulatory network. An additional approach instead proposed to maximize a penalized likelihood for constructing a sparse gene regulatory network.

Elucidating relationships between genes, and the products they encode, remains one of the central challenges in experimental and computational biology. A gene regulatory network (GRN) is a directed graph in which regulators of gene expression are connected to target gene nodes by interaction edges. Regulators of gene expression include transcription factors (TF) which can act as activators and repressors, RNA binding proteins, and regulatory RNAs. Identifying regulatory relationships between transcriptional regulators and their targets is essential for understanding biological phenomena ranging from cell growth and division to cell differentiation and development. Reconstruction of GRNs is required to understand how gene expression dysregulation contributes to cancer and complex heritable diseases.

Genome-scale methods provide an efficient means of identifying gene regulatory relationships. Efforts of the past two decades have resulted in the development of a variety of experimental and computational methods that leverage advances in technology and machine learning for constructing GRNs. This method takes as inputs gene expression data and sources of prior information, and outputs regulatory relationships between transcription factors and their target genes that explain the observed gene expression levels. Subsequent work has enhanced this approach by selecting regulators for each gene more effectively, incorporating orthogonal data types that can be used to generate constraints on network structure, and explicitly estimating latent biophysical parameters including transcription factor activity and mRNA decay rates.

Recent advances in sequencing technologies make it feasible to obtain both whole-genome genotype and gene expression for one or more target genes and/or one or more species and/or one or more subjects, i.e., genetical genomics data. Combining genetics with gene expression reveals additional information on genetic structure and holds great promise for improving the accuracy of gene regulatory network inference. Numerous genetical genomics experiments, such as the Genotype-Tissue Expression (GTEx) project, have been conducted to collect genetical genomics data.

Much effort has been devoted to using genetical genomics data for genome-wide association (GWA) analysis of gene expression, i.e., expression quantitative trait loci (eQTL) mapping. Mapping of eQTL intends to elucidate variation of expression traits attributed to genomic variation, and to identify chromosomal loci (i.e., eQTL) of genetic polymorphisms associated to the expression of a gene under investigation. An eQTL located within the region of the gene under investigation is called a cis-eQTL, otherwise it is called a trans-eQTL. While the cis effects of a gene represent direct regulations, indirect regulations of trans-eQTL are likely caused by interactions among genes. These eQTL provide insight on the functional sequences of the gene expression, and thus an indirect interrogation of the functional landscape of gene regulations.

In an aspect, “RNA Editing” can refer to a type of genetic engineering in which an RNA molecule (or ribonucleotides of the RNA) is inserted, deleted, or replaced in the genome of an organism using engineered nucleases, which create site-specific strand breaks at desired locations in the RNA. The induced breaks are repaired resulting in targeted mutations or repairs.

In an aspect, “CRISPR-based endonucleases” include RNA-guided endonucleases that comprise at least one nuclease domain and at least one domain that interacts with a guide RNA. As known to the art, a guide RNA directs the CRISPR-based endonucleases to a targeted site in a nucleic acid at which site the CRISPR-based endonucleases cleaves at least one strand of the targeted nucleic acid sequence. As the guide RNA provides the specificity for the targeted cleavage, the CRISPR-based endonuclease is universal and can be used with different guide RNAs to cleave different target nucleic acid sequences. CRISPR-based endonucleases are RNA-guided endonucleases derived from CRISPR/Cas systems. Guide RNAs can be crafted to target any gene, for example, or any other target gene. In an aspect, a disclosed gRNA can comprise 2 parts: (i) crispr RNA (crRNA), which is a 17-20 nucleotide sequence complementary to the targeted DNA or targeted locus, and (ii) a tracr RNA, which serves as a binding scaffold for a Cas nuclease. In an aspect, a targeted DNA or targeted locus can be a gene having one or more mutations or defects that contribute to one or more genetic diseases or disorders.

In an aspect, a disclosed CRISPR-based endonuclease can be derived from a CRISPR/Cas type I, type II, or type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

In an aspect, a disclosed CRISPR-based endonuclease can be derived from a type II CRISPR/Cas system. For example, in an aspect, a CRISPR-based endonuclease can be derived from a Cas9 protein. The Cas9 protein can be from Streptococcus pyogenes, Streptococcus thermophilus, Streptococcus sp, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. In an aspect, the CRISPR-based nuclease can be derived from a Cas9 protein from Streptococcus pyogenes.

In an aspect, “dCas9” refers to enzymatically inactive form of Cas9, which can bind, but cannot cleave, DNA. In an aspect, a disclosed variant Cas9 can comprise VQR, EQR, or VRER.

In an aspect, “Protospacer Adjacent Motif” or “PAM” refers to a sequence adjacent to the target sequence that is necessary for Cas enzymes to bind target DNA.

A “protospacer sequence” refers to the target double stranded DNA and specifically to the portion of the target DNA (e.g., or target region in the genome) that is fully or substantially complementary (and hybridizes) to the spacer sequence of the CRISPR arrays. The protospacer sequence in a Type I system is directly flanked at the 3′ end by a PAM. A spacer is designed to be complementary to the protospacer.

In general, a gRNA (also referred to herein as “gRNA scaffold” interchangeably) can complex with a compatible nucleic acid-guided nuclease and can hybridize with a target sequence, thereby directing the nuclease to the target sequence. A subject nucleic acid-guided nuclease capable of complexing with a guide polynucleotide can be referred to as a nucleic acid-guided nuclease that is compatible with the gRNA. In addition, a gRNA capable of complexing with a nucleic acid-guided nuclease can be referred to as a guide polynucleotide or a guide nucleic acid that is compatible with the nucleic acid-guided nucleases.

A gRNA can include a scaffold sequence. In general, a “scaffold sequence” can include any sequence that has sufficient sequence to promote formation of a targetable nuclease complex, wherein the targetable nuclease complex includes, but is not limited to, a nucleic acid-guided nuclease and a guide polynucleotide can include a scaffold sequence and a guide sequence. Sufficient sequence within the scaffold sequence to promote formation of a targetable nuclease complex can include a degree of complementarity along the length of two sequence regions within the scaffold sequence, such as one or two sequence regions involved in forming a secondary structure. In an aspect, the one or two sequence regions are included or encoded on the same polynucleotide. In an aspect, the one or two sequence regions are included or encoded on separate polynucleotides. Optimal alignment can be determined by any suitable alignment algorithm, and can further account for secondary structures, such as self-complementarity within either the one or two sequence regions. In an aspect, the degree of complementarity between the one or two sequence regions along the length of the shorter of the two when optimally aligned can be about or more than about 25%, 30%, 40%, 50%, 60%, 70%, 80%, 90%, 95%, 97.5%, 99%, or higher. In an aspect, at least one of the two sequence regions can be about or more than about 5, 6, 7, 8, 9, 10, 11, 12, 13, 14, 15, 16, 17, 18, 19, 20, 25, 30, 40, 50, or more nucleotides in length.

A scaffold sequence of a subject guide polynucleotide can comprise a secondary structure. A secondary structure can comprise a pseudoknot region. In an aspect, binding kinetics of a guide polynucleotide to a nucleic acid-guided nuclease is determined in part by secondary structures within the scaffold sequence. In an aspect, binding kinetics of a guide polynucleotide to a nucleic acid-guided nuclease is determined in part by nucleic acid sequence with the scaffold sequence.

Gene editing refers to a type of genetic engineering in which the nucleotide sequence of a target polynucleotide is changed through introduction of deletions, insertions, or base substitutions to the polynucleotide sequence. In some aspects, CRISPR-mediated gene editing utilizes the pathways of non-homologous end-joining (NHEJ) or homologous recombination to perform the edits. Gene regulation refers to increasing or decreasing the production of specific gene products such as protein or RNA.

In an aspect, “homology directed repair” or “HDR” can occur either non-conservatively or conservatively. The non-conservative method is composed of the single-strand annealing (SSA) pathway and is more error prone. The conservative methods, characterized by the accurate repair of the DSB by means of a homologous donor (e.g., sister chromatid, plasmid, etc.), are composed of three pathways: the classical double-strand break repair (DSBR), synthesis-dependent strand-annealing (SDSA), and break-induced repair (BIR). For example, in the classical DSBR pathway, the 3′ ends invade an intact homologous template to serve as a primer for DNA repair synthesis, ultimately leading to the formation of double Holliday junctions (dHJs). dHJs are four-stranded branched structures that form when elongation of the invasive strand “captures” and synthesizes DNA from the second DSB end. The individual HJs are resolved via cleavage in one of two ways. Each junction resolution could happen on the crossing strand (horizontally at the purple arrows) or on the non-crossing strand (vertically at the orange arrows). If resolved dissimilarly (e.g., one junction is resolved on the crossing strand and the other on the non-crossing strand), then a crossover event will occur; however, if both HJs are resolved in the same manner, this results in a non-crossover event. DSBR is semi-conservative, as crossover events are most common.

In an aspect, a disclosed method can comprise validating a CRISPR event. In an aspect, validation of a CRISPR event can be accomplished using methods and techniques known to the art (e.g., sequencing, northern blots, FISH, PCR, RNA-Seq, 3′ RACE, 5′ RACE, etc.).

In an aspect, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides can be collectively referred to as “gene product.” If the polynucleotide is derived from genomic DNA, then expression can include splicing of the mRNA in a eukaryotic cell.

In an aspect, “target molecule” refers to a molecule of interest, the amount or expression level of which is directly or indirectly influenced by the activity of a fusion protein comprising the protein of interest fused in-frame with maltose-dependent degradation determinants. In an aspect, the term “target molecule” can refer to, for example, enzymes, other proteins, peptides, amino acids, nucleic acids, lipids, carbohydrates, metabolites, and non-catabolic compounds.

In an aspect, “functionally disrupted” or “functional disruption” of a selected gene can refer to an alteration of the selected gene in such a way that the activity of the protein encoded by the selected gene in the host cell is reduced. In an aspect, “functional disruption” or “functional disruption” of a selected protein means that the protein is altered in such a way that the activity of the protein in the host cell is reduced. In an aspect, the activity of the selected protein encoded by the selected gene is abolished in the host cell. In an aspect, the activity of the selected protein encoded by the selected gene is reduced in a host cell. In an aspect, functional disruption of the selected gene can be achieved by deleting all or part of the gene, thereby eliminating or reducing gene expression, or eliminating or reducing the activity of the gene product. In an aspect, functional disruption of the selected gene may also be achieved by mutating the regulatory elements of the gene, for example the promoter of the gene, thereby eliminating or reducing expression, or by mutating the coding sequence of the gene, such that the activity of the gene product is eliminated or reduced. In an aspect, the functional disruption of the selected gene results in the removal of the entire open reading frame of the selected gene.

In an aspect, “introduced” refers to the introduction by means of modern biotechnology, and not a naturally occurring introduction.

In an aspect, “introduced genetic material” means genetic material that is added to, and remains as a component of, the genome of the recipient.

In an aspect, the term “native” or “endogenous” refers to a substance or process/method that may occur naturally in a host cell.

In an aspect, “genetically modified” denotes a host cell (such as, for example, a cell having one or more genetic modifications) comprising a heterologous nucleotide sequence.

In an aspect, “isolated nucleic acid” when applied to DNA refers to a DNA molecule that is isolated from the immediate sequence in the naturally occurring genome of the organism from which it originates. In an aspect, an “isolated nucleic acid” also includes non-genomic nucleic acids, such as cDNA or other non-naturally occurring nucleic acid molecules.

In an aspect, the term “cDNA” is a DNA molecule that can be made by reverse transcription from a mature, spliced mRNA molecule obtained from a cell. cDNA lacks intron sequences that are normally present in the corresponding genomic DNA.

In an aspect, “operably linked” refers to a functional linkage between nucleic acid sequences such that the linked promoter and/or regulatory region functionally controls the expression of the coding sequence.

In an aspect, “epigenome modification” refers to a modification or change in one or more chromosomes that affect gene activity and expression that does not derive from a modification of the genome. An epigenome modification relates to a functionally relevant change to the genome that does not involve a change in the nucleotide sequence. Epigenome modifications may include a modification to a histone, such as acetylation, methylation, phosphorylation, ubiquitination, and/or sumoylation. Epigenome modifications may include a modification to DNA, such as methylation.

In an aspect, “biosynthetic pathway” refers to a pathway having a set of anabolic or catabolic biochemical reactions for converting one chemical into another, thereby producing a molecule. Gene products belong to the same “biosynthetic pathway” if they act in parallel or in series on the same substrate, producing the same product, or on or producing a metabolic intermediate (e.g., metabolite) between the same substrate and the metabolite end product.

In an aspect, “biosynthetic pathway” refers to a pathway having a set of anabolic or catabolic biochemical reactions for converting one chemical into another, thereby producing a molecule. Gene products belong to the same “biosynthetic pathway” if they act in parallel or in series on the same substrate, producing the same product, or on or producing a metabolic intermediate (e.g., metabolite) between the same substrate and the metabolite end product.

In an aspect, “promoter” refers to a nucleic acid of synthetic or natural origin which is capable of conferring, activating or enhancing the expression of a DNA coding sequence. A promoter may comprise one or more specific transcriptional regulatory sequences to further enhance expression and/or alter spatial and/or temporal expression of the coding sequence. The promoter can be located 5′ (upstream) of the coding sequence under its control. The distance between the promoter and the coding sequence to be expressed can be about the same as the distance between the promoter and the native nucleic acid sequence it controls. Variations in the distance can be accommodated without loss of promoter function.

In an aspect, “transcriptional regulator” refers to a protein that controls gene expression. In an aspect, “transcriptional activator” refers to a transcriptional regulator that activates or positively regulates gene expression. In an aspect, “transcription repressing factor” refers to a transcription regulatory factor that represses or negatively regulates gene expression.

In an aspect, “gene that affects cell growth” or “nucleic acid encoding a protein that affects cell growth” refers to a nucleic acid that encodes a protein that affects cell growth (e.g., growth rate or cell biomass) of a cell.

In an aspect, “essential gene” refers to a gene absolutely required to sustain life under optimal conditions where all nutrients are available. In an aspect, “conditionally essential gene” refers to a gene that is only essential under certain conditions or growth conditions.

In an aspect, “regulator” refers to a genome or group of nucleic acids that is regulated by the same regulatory protein (e.g., a transcriptional regulator). The genes of the regulon have regulatory binding sites or have promoters that are regulated by common transcriptional regulators. The genome or nucleic acid set comprising the regulon can be located continuously or discontinuously in the genome of the host cell.

In an aspect, “inducible promoter” refers to a promoter that is activated by an inducer to induce transcription of a gene it controls.

In an aspect, “constitutive promoter” refers to a promoter that does not require the presence of an inducer to induce transcription of the gene it controls.

In an aspect, “expression” refers to the production of mRNA by transcription of the gene of interest and/or by transcription of the gene to produce a protein, followed by translation of the mRNA.

In an aspect, “catabolism” refers to a process/method of molecular breakdown or degradation of a large molecule into smaller molecules.

In an aspect, “non-catabolic” refers to processes/methods of constructing molecules from smaller units, and these reactions typically require energy. The term “non-catabolic compound” refers to a compound produced by a non-catabolic process.

In an aspect, a target gene can be inaccessible to one or more transcriptional control sequences.

In an aspect, a transcriptional control sequence can regulate, modulate, or influence expression of one or more target genes.

In an aspect, a disclosed target gene can have a defined state of expression, e.g., expression in its native state and/or expression in a diseased state. In an aspect, a disclosed target gene can have a moderate to low level of expression. In an aspect, a disclosed target gene can have a moderate to high level of expression.

In an aspect, the targeting moiety targets one or more nucleotides, e.g., such as through CRISPR, TALEN, dCas9, oligonucleotide pairing, recombination, transposon, etc., of a gene targeted for gene expression by, for example, substitution, addition, or deletion. TALENs are a genome editing method derived from plant pathogenic bacteria. TALE architecture is composed of three parts: an N-terminal domain, TALE repeat domains, and a C-terminal domain. The TALE repeat domains typically consist of 34 amino acid residues, where the 12th and 13th repeat variable di-residues (RVDs) determine DNA nucleotide binding specificity. Each RVD recognizes a specific nucleotide, leading to a simple code for DNA recognition: NI for adenine, HD for cytosine, NG for thymine and NH or NN for guanine. Importantly, the RVDs can be assembled sequentially to bind any given target sequence. For genome editing purposes, TALEs are fused to the FokI nuclease domain to create TALE nucleases (TALENs). Because FokI only cleaves as a dimer, sites must be targeted by a pair of TALENs binding on opposite faces of the DNA strand, spaced ~14-20 bp apart. The FokI nuclease domains dimerize across the spacer sequence and create a double-strand break (DSB). The DSB can be repaired through error-prone non-homologous end-joining (NHEJ), which often results in indels and potentially frameshift mutations. For efficient binding, TALEN target sequences require a thymine at the 5′ end for recognition by the TALE N-terminus.

In an aspect, a disclosed targeting moiety can alter one or more nucleotides, such as through a gene editing system, of a sequence in a gene targeted for gene expression by, for example, substitution, addition or deletion.

In an aspect, the activity or expression of target gene can be modulated. In an aspect, modulated means increased or decreased expression of a target gene during any point before, after, or during translation.

In an aspect, activity or expression of a disclosed target gene can be modulated during translation. In an aspect, inhibition of translation of a disclosed target gene can be modulated expression. In an aspect, the expression level of a disclosed target gene can be modulated if the steady-state level of the expressed protein decreased even though translation was not inhibited.

In an aspect, a change in the half-life of a mRNA can modulate expression. In an aspect, modulated activity or expression of a target gene can be increased or decreased expression during any point before, during, or after translation.

In an aspect, the activity or expression of a disclosed target gene can be increased by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 20%, or more than at least about 100% following contact and/or exposure with a modulatory technology (e.g., CRISPR, TALEN, dCas9, oligonucleotide pairing, recombination, transposon, etc.).

In an aspect, the activity or expression of a disclosed target gene can be decreased by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 20%, or more than at least about 100% following contact and/or exposure with a modulatory technology (e.g., CRISPR, TALEN, dCas9, oligonucleotide pairing, recombination, transposon, etc.).

In an aspect, the activity or expression of a disclosed target gene can be increased by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 20%, or more than at least about 100% following contact and/or exposure with a modulatory technology (e.g., CRISPR, TALEN, dCas9, oligonucleotide pairing, recombination, transposon, etc.) when compared to a non-modulated level of activity or expression (e.g., having no contact and/or exposure with a modulatory technology).

In an aspect, the activity or expression of a disclosed target gene can be decreased by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 20%, or more than at least about 100% following contact and/or exposure with a modulatory technology (e.g., CRISPR, TALEN, dCas9, oligonucleotide pairing, recombination, transposon, etc.) when compared to a non-modulated level of activity or expression (e.g., having no contact and/or exposure with a modulatory technology).

In an aspect, upregulate is a process by which a cell increases its response to a substance or signal from outside the cell to carry out a specific function. In an aspect downregulate is a process of reducing or suppressing a response to a stimulus.

As known to the skilled person, CHOPCHOP is a versatile tool for selecting target sites for CRISPR (Cas9, Cas9 nickase, Cas13, Cpf1/CasX) or TALEN-directed mutagenesis. The V3 release of CHOPCHOP includes many new features and visualization options, supporting over 200 genomes with whole-gene targeting and versatile search modes. The tool now enables precise RNA targeting with Cas13, supporting alternative transcript isoforms and predicting RNA accessibility using ViennaRNA's RNAfold. The update also introduces new DNA targeting modes, including CRISPR activation/repression, targeted enrichment of loci for long-read sequencing, and prediction of Cas9 repair outcomes. For larger queries or handling unsupported genomes, a command-line version is available with additional functionalities. CHOPCHOP V3 is user-friendly and well-equipped to support diverse targeting applications, facilitating effective experimental design.

In an aspect, numerous bacterial nucleic acid modification systems have led to the recent development of two modular, precise genome editing tools. The TALE (transcription activator-like effector) and CRISPR/Cas (clustered regularly interspaced short palindromic repeats) systems have recently been optimized for research use to site-specifically introduce mutations and manipulate transcriptional activation and repression in a variety of organisms.

TALENs are a genome editing method derived from plant pathogenic bacteria. TALE architecture is composed of three parts: an N-terminal domain, TALE repeat domains, and a C-terminal domain. The TALE repeat domains typically consist of 34 amino acid residues, where the 12th and 13th repeat variable di-residues (RVDs) determine DNA nucleotide binding specificity. Each RVD recognizes a specific nucleotide, leading to a simple code for DNA recognition: NI for adenine, HD for cytosine, NG for thymine and NH or NN for guanine. Importantly, the RVDs can be assembled sequentially to bind any given target sequence. For genome editing purposes, TALEs are fused to the FokI nuclease domain to create TALE nucleases (TALENs). Because FokI only cleaves as a dimer, sites must be targeted by a pair of TALENs binding on opposite faces of the DNA strand, spaced ~14-20 bp apart. The FokI nuclease domains dimerize across the spacer sequence and create a double-strand break (DSB). The DSB can be repaired through error-prone non-homologous end-joining (NHEJ), which often results in indels and potentially frameshift mutations. For efficient binding, TALEN target sequences require a thymine at the 5′ end for recognition by the TALE N-terminus.

The CRISPR/Cas9 system originates from a bacterial immune system that has been adopted for use as a programmable genome editing tool. Streptococcus pyogenes Cas9 nuclease is directed to target sites in the genome by a single-guide RNA (sgRNA). The Cas9/sgRNA complex binds a 20 bp target sequence followed by a 3 bp protospacer adjacent motif (PAM) −NGG (two invariable Gs preceded by a variable base), and it creates a DSB that is repaired in a seemingly identical manner to TALEN-induced DSBs. While the presence of an −NGG PAM motif is one of the few requirements for binding, the methods used to generate sgRNAs for targeting often impose additional restrictions. Depending on the polymerase used for sgRNA synthesis, the 5′ end dinucleotides can be limited to, for example, 5′ GN− for the commonly used U6 promoter (polymerase III), or 5′ GG− for T7 polymerase. In addition, certain criteria such as guanine-cytosine content (GC-content) appear to influence binding efficiency. These, along with other guidelines to ensure target suitability, have been used to mostly manually design sgRNAs to generate mutations and knockouts in a variety of organisms including bacteria, yeast, zebrafish, Xenopus, nematodes, fruit flies, mice and human cells.

TALEN and sgRNA design require identification of target sites that fulfill certain sequence requirements while simultaneously avoiding off-targets elsewhere in the genome. Several studies have demonstrated the limited specificity of TALEN—and particularly Cas9-based genome editing strategies, highlighting the importance of determining the uniqueness of each candidate target site. Existing tools for identifying TALEN or sgRNA target sites have limitations, including acceptance of few input formats, slow search times, restriction to either TALEN or CRISPR/Cas9 target design, minimal or no visualization of the target locus and/or limited information about potential off-target sites.

The method according to any embodiment or aspect described above, wherein validating the selection of the engineered organism comprises measuring the expression of one or more genes of the engineered organism.

The method according to any embodiment or aspect described above, further comprising repeating the measuring the expression of one or more genes of the engineered organism.

The method according to any embodiment or aspect described above, wherein measuring gene expression can comprise using high-density expression array, DNA microarray, polymerase chain reaction (PCR), reverse transcriptase PCR (RT-PCR), real-time quantitative reverse transcription PCR (qRT-PCR), serial analysis of gene expression (SAGE), spotted cDNA arrays, GeneChip, spotted oligo arrays, bead arrays, RNA Seq, tiling array, northern blotting, hybridization microarray, in situ hybridization, whole-exome sequencing, whole-genome sequencing, liquid biopsy, next-generation sequencing, or any combination thereof.

In an aspect, “determining” can refer to measuring or ascertaining the expression of one or more genes. By “determining the amount” is meant both an absolute quantification of a particular analyte or a determination of the relative abundance of a particular analyte (e.g., a target gene or a gene of interest). The phrase includes both direct or indirect measurements of abundance or both.

In an aspect, measuring the expression of a disclosed transgene and/or the reporter gene can comprise measuring the protein concentration of the transgene and/or the reporter gene or measuring the mRNA level of transgene and/or the reporter gene. For example, in an aspect, measuring the protein concentration of transgene and/or the reporter gene comprises a protein chip analysis, an immunoassay, a ligand binding assay, a MALDI-TOF (Matrix Assisted Laser Desorption/Ionization Time of Flight Mass Spectrometry) analysis, a SELDI-TOF (Sulface Enhanced Laser Desorption/Ionization Time of Flight Mass Spectrometry) analysis, a radioimmunoassay, a radioimmunodiffusion assay, an octeroni immunodiffusion method, rocket immunoelectrophoresis, tissue immunostaining, a complement fixation assay, 2D by electrophoretic analysis, liquid chromatography-Mass Spectrometry (LC-MS), liquid chromatography-Mass Spectrometry/Mass Spectrometry (LC-MS/MS), Western blotting, ELISA (enzyme linked immunosorbent assay), or any combination thereof. Similarly, in an aspect, measuring the mRNA level of a targeted gene and/or the reporter gene comprises a reverse transcription polymerase reaction (RT-PCR), a competitive reverse transcription polymerase reaction (Competitive RT-PCR), a real-time reverse transcription polymerization, an enzyme reaction (Real-time RT-PCR), an RNase protection assay (RPA), Northern blotting, a DNA chip, or any combination thereof.

The terms “transformation,” “transfection,” and “transduction” as used interchangeably herein refer to the introduction of a heterologous nucleic acid molecule into a cell. Such introduction into a cell can be stable or transient. Thus, in an aspect, a host cell or host organism is stably transformed with a polynucleotide of the disclosure. In an aspect, a host cell or host organism is transiently transformed with a polynucleotide of the disclosure. “Transient transformation” in the context of a polynucleotide means that a polynucleotide is introduced into the cell and does not integrate into the genome of the cell. By “stably introducing” or “stably introduced” in the context of a polynucleotide introduced into a cell is intended that the introduced polynucleotide is stably incorporated into the genome of the cell, and thus the cell is stably transformed with the polynucleotide. In an aspect, “stable transformation” or “stably transformed” means that a nucleic acid molecule is introduced into a cell and integrates into the genome of the cell. As such, the integrated nucleic acid molecule is capable of being inherited by the progeny thereof, more particularly, by the progeny of multiple successive generations. In an aspect, “genome” also includes the nuclear, the plasmid and the plastid genome, and therefore includes integration of the nucleic acid construct into, for example, the chloroplast or mitochondrial genome. In an aspect, stable transformation can also refer to a transgene that is maintained extra-chromosomally, for example, as a mini-chromosome or a plasmid. In an aspect, the nucleotide sequences, constructs, expression cassettes can be expressed transiently and/or they can be stably incorporated into the genome of the host organism.

In an aspect, the terms “increase,” “increasing,” “increased,” “enhance,” “enhanced,” “enhancing,” and “enhancement” (and grammatical variations thereof) describe an elevation of at least about 25%, 50%, 75%, 100%, 150%, 200%, 300%, 400%, 500% or more as compared to a control level or reference level (e.g., increased expression, increased titer, increased expression capacity, increased packaging capacity, increased transduction efficiency, or any combination thereof).

In an aspect, the terms “decrease”, “decreasing”, “decreased”, “diminish”, “diminished”, “diminishing”, and “diminishment” (and grammatical variations thereof) describe a decrease of at least about 25%, 50%, 75%, 100%, 150%, 200%, 300%, 400%, 500% or more as compared to a control level or a reference level (e.g., decreased expression, decreased titer, decreased expression capacity, decreased packaging capacity, decreased transduction efficiency, or any combination thereof).

In an aspect, “transgene” refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism's genetic code. The introduction of a transgene has the potential to change the phenotype of an organism.

In genetics, gene-gene interaction (epistasis) is the effect of one gene on a disease modified by another gene or several other genes. Biological epistasis, i.e., the gene-gene interaction has biological basis, is in contrast to statistical epistasis that describes deviation from additivity in a linear statistical model. Epistasis can be contrasted with dominance, which is an interaction between alleles at the same gene locus. Gene-gene interaction is a common component of genetic architecture of human complex diseases; however, it is difficult to detect. The multilocus genotype combinations for gene-gene interaction increase exponentially and require larger sample size as well as more computation burden. The commonly used linear models have limited ability to detect nonlinear patterns of gene-gene interaction.

### Examples of Digenic Epistatic Ratios

| Ratio | Description | Name of Relationship |
|---|---|---|
| 9:3:3:1 | Complete dominance at both gene pairs; new | Not named because the ratio |
| | phenotypes result from interaction between | looks like independent |
| | dominant alleles, as well as from interaction | assortment |
| | between both homozygous recessives | |
| 9:4:3 | Complete dominance at both gene pairs; | Recessive epistasis |
| | however, when one gene is homozygous | |
| | recessive, it hides the phenotype of the other | |
| | gene | |
| 9:7 | Complete dominance at both gene pairs; | Duplicate recessive epistasis |
| | however, when either gene is homozygous | |
| | recessive, it hides the effect of the other gene | |
| 12:3:1 | Complete dominance at both gene pairs; | Dominant epistasis |
| | however, when one gene is dominant, it hides | |
| | the phenotype of the other gene | |
| 15:1 | Complete dominance at both gene pairs; | Duplicate dominant epistasis |
| | however, when either gene is dominant, it | |
| | hides the effects of the other gene | |
| 13:3 | Complete dominance at both gene pairs; | Dominant and recessive |
| | however, when either gene is dominant, it | epistasis |
| | hides the effects of the other gene | |
| 9:6:1 | Complete dominance at both gene pairs; | Duplicate interaction |
| | however, when either gene is dominant, it | |
| | hides the effects of the other gene | |
| 7:6:3 | Complete dominance at one gene pair and | No name |
| | partial dominance at the other; when | |
| | homozygous recessive, the first gene is | |
| | epistatic to the second gene | |
| 3:6:3:4 | Complete dominance at one gene pair and | No name |
| | partial dominance at the other; when | |
| | homozygous recessive, either gene hides the | |
| | effects of the other gene; when both genes are | |
| | homozygous recessive, the second gene hides | |
| | the effects of the first | |
| 11:5 | Complete dominance for both gene pairs only | No name |
| | if both kinds of dominant alleles are present; | |
| | otherwise, the recessive phenotype appears | |

Although certain examples have been illustrated and described herein for purposes of description, a wide variety of alternate and/or equivalent examples or implementations calculated to achieve the same purposes may be substituted for the examples shown and described without departing from the scope of the present disclosure. This application is intended to cover any adaptations or variations of the examples discussed herein, including the implementation or utilization of components of the systems or steps independently and separately from other described components or steps. Therefore, it is manifestly intended that examples described herein be limited only by the claims.

### LISTING OF SEQUENCES

| SEQ ID NO | Description |
|---|---|
| 1 | ADE1/YAR015W (Saccharomyces cerevisiae) - Protein |
| 2 | ADE1/YAR015W (Saccharomyces cerevisiae) - Genomic |
| 3 | Cas9 (Streptococcus pyogenes) - Protein |
| 4 | Cas9 (Streptococcus pyogenes) - DNA |
| 5 | Cas9 (Staphylococcus Aureus) - Protein |
| 6 | Cas9 (Staphylococcus Aureus) - DNA |

What is claimed is:

1. A method of determining gene to gene interaction based upon gene modification, the method comprising:
extracting a cooperative network implementing an unsupervised regression tree;
classifying a plurality of gene interactions into one of an up-regulation category and/or a down-regulation category;
creating a gene expression profile matrix based upon gene data and resulting expression weighting functions;
receiving a request to modify a gene and/or an expression of the gene; and
simulating an interaction of the modification within the gene expression profile matrix to provide a weighted direction graph.

2. The method of claim 1, wherein the unsupervised regression trees include unsupervised random forests and decision trees for axis unimodal clustering.

3. The method of claim 1, wherein classifying the plurality of gene interactions into one of the up-regulation category and/or the down-regulation category is based upon one or more predictor genes in a model for a target gene.

4. The method of claim 3, further comprising generating a random sample of a first subset in which an expression of a predictor gene is higher than a median expression, and generating a random sample of a second subset in which an expression of the predictor gene is lower than the median expression.

5. The method of claim 4, further comprising predicting a first value for the first subset and predicting a second value for the second subset using the unsupervised regression trees;
inferring a positive interaction when the first value is higher than the second value; and
inferring a negative interaction when the second value is higher the first value.

6. The method of claim 5, further comprising repeating, for each target gene and predictor gene combination, the generating of the random sample of the first subset, the generating of the random sample of the second subset, predicting the first value, predicting the second value, and inferring one of a positive or negative interaction.

7. The method of claim 6, further comprising determining, based on real-time existing data, a rate of change of expression levels corresponding to the positive or negative interaction of all the target genes and predictor genes on one another; suggesting a gene editing strategy based upon the rate of change of expression levels.

8. A system of determining gene to gene interaction based upon gene modification, the system comprising:
at least one memory configured to store machine executable instructions; and
at least one processor communicatively coupled with the at least one memory and configured to execute the machine executable instructions to perform operations comprising:
extracting a cooperative network implementing an unsupervised regression tree;
classifying a plurality of gene interactions into one of an up-regulation category and/or a down-regulation category;
creating a gene expression profile matrix based upon gene data and resulting expression weighting functions;
receiving a request to modify a gene and/or an expression of the gene; and
simulating an interaction of the modification within the gene expression profile matrix to provide a weighted direction graph.

9. The system of claim 8, wherein the unsupervised regression trees include unsupervised random forests and decision trees for axis unimodal clustering.

10. The system of claim 8, wherein classifying the plurality of gene interactions into one of the up-regulation category and/or the down-regulation category is based upon one or more predictor genes in a model for a target gene.

11. The system of claim 10, wherein the operations further comprising generating a random sample of a first subset in which an expression of a predictor gene is higher than a median expression, and generating a random sample of a second subset in which an expression of the predictor gene is lower than the median expression.

12. The system of claim 11, wherein the operations further comprising predicting a first value for the first subset and predicting a second value for the second subset using the unsupervised regression trees; inferring a positive interaction when the first value is higher than the second value; and inferring a negative interaction when the second value is higher the first value.

13. The system of claim 12, wherein the operations further comprising repeating, for each target gene and predictor gene combination, the generating of the random sample of the first subset, the generating of the random sample of the second subset, predicting the first value, predicting the second value, and inferring one of a positive or negative interaction.

14. The system of claim 13, wherein the operations further comprising determining, based on real-time existing data, a rate of change of expression levels corresponding to the positive or negative interaction of all the target genes and predictor genes on one another; suggesting a gene editing strategy based upon the rate of change of expression levels.

15. A non-transitory computer-readable medium (CRM) comprising machine-executable instructions stored thereon, which, when executed by at least one processor of a computing device, cause the computing device to determine gene to gene interaction based upon gene modification by performing operations comprising:
extracting a cooperative network implementing an unsupervised regression tree;
classifying a plurality of gene interactions into one of an up-regulation category and/or a down-regulation category;
creating a gene expression profile matrix based upon gene data and resulting expression weighting functions;
receiving a request to modify a gene and/or an expression of the gene; and
simulating an interaction of the modification within the gene expression profile matrix to provide a weighted direction graph.

16. The non-transitory CRM of claim 15, wherein the unsupervised regression trees include unsupervised random forests and decision trees for axis unimodal clustering.

17. The non-transitory CRM of claim 15, wherein classifying the plurality of gene interactions into one of the up-regulation category and/or the down-regulation category is based upon one or more predictor genes in a model for a target gene.

18. The non-transitory CRM of claim 17, wherein the operations further comprising generating a random sample of a first subset in which an expression of a predictor gene is higher than a median expression, and generating a random sample of a second subset in which an expression of the predictor gene is lower than the median expression.

19. The non-transitory CRM of claim 18, wherein the operations further comprising predicting a first value for the first subset and predicting a second value for the second subset using the unsupervised regression trees; inferring a positive interaction when the first value is higher than the second value; and inferring a negative interaction when the second value is higher the first value.

20. The non-transitory CRM of claim 19, wherein the operations further comprising:
repeating, for each target gene and predictor gene combination, the generating of the random sample of the first subset, the generating of the random sample of the second subset, predicting the first value, predicting the second value, and inferring one of a positive or negative interaction; or
determining, based on real-time existing data, a rate of change of expression levels corresponding to the positive or negative interaction of all the target genes and predictor genes on one another; suggesting a gene editing strategy based upon the rate of change of expression levels.


SEQUENCE LISTING (6 sequences)

SEQ ID NO: 1 - protein, 306 aa, Saccharomyces cerevisiae
MSITKTELDGILPLVARGKVRDIYEVDAGTLLFVATDRISAYDVIMENSIPEKGILLTKLSEFWFKFLSNDVRNHLVDIAPGKTIFDYLPAKLSEPKYKTQLEDRSLLVHKHKLIPLEVIVRGYITGSAWKEYVKTGTVHGLKQPQGLKESQEFPEPIFTPSTKAEQGEHDENISPAQAAELVGEDLSRRVAELAVKLYSKCKDYAKEKGIIIADTKFEFGIDEKTNEIILVDEVLTPDSSRFWNGASYKVGESQDSYDKQFLRDWLTANKLNGVNGVKMPQDIVDRTRAKYIEAYETLTGSKWSH

SEQ ID NO: 2 - DNA, 921 bp, Saccharomyces cerevisiae
  source 1..921: mol_type = genomic DNA
atgtcaattacgaagactgaactggacggtatattgccattggtggccagaggtaaagttagagacatatatgaggtagacgctggtacgttgctgtttgttgctacggatcgtatctctgcatatgacgttattatggaaaacagcattcctgaaaaggggatcctattgaccaaactgtcagagttctggttcaagttcctgtccaacgatgttcgtaatcatttggtcgacatcgccccaggtaagactattttcgattatctacctgcaaaattgagcgaaccaaagtacaaaacgcaactagaagaccgctctctattggttcacaaacataaactaattccattggaagtaattgtcagaggctacatcaccggatctgcttggaaagagtacgtaaaaacaggtactgtgcatggtttgaaacaacctcaaggacttaaagaatctcaagagttcccagaaccaatcttcaccccatcgaccaaggctgaacaaggtgaacatgacgaaaacatctctcctgcccaggccgctgagctggtgggtgaagatttgtcacgtagagtggcagaactggctgtaaaactgtactccaagtgcaaagattatgctaaggagaagggcatcatcatcgcagacactaaattcgaattcggtattgacgaaaagaccaatgaaattattctagtggacgaggtgctaacgccagactcctctagattctggaacggtgcctcttataaggtaggagaatcccaagattcttacgataagcaatttttaagagactggcttactgctaataagttgaacggtgttaacggcgtcaaaatgccccaagacattgtcgacaggacaagggccaaatatatagaggcttatgaaacattgacagggtctaaatggtctcactaa

SEQ ID NO: 3 - DNA, 3162 bp, Staphylococcus aureus
  source 1..3162: mol_type = genomic DNA
atgaaaaggaattatatcttaggattagatatcggaattacatcagtgggttatggaattattgattatgaaactagagatgtcatagatgcgggcgtacgtttatttaaagaggctaatgttgaaaataatgaaggacgacgatcaaaaagaggtgccagaaggcttaagaggcgtcgtagacatagaatacaaagagtaaagaaacttttatttgattacaatttgttgacagatcatagtgagctaagtggaatcaatccttacgaggcgcgcgtaaagggattaagtcaaaaattaagtgaagaggaattttctgcggcattgctacatttagcaaagcgtagaggtgtacataatgttaatgaagtggaagaagatacaggtaatgaattatccactaaagaacaaatttcaagaaatagtaaagcgttagaagagaagtatgttgcagaattacagttggaacgtttgaaaaaagacggtgaagtgagaggttcgattaaccgtttcaaaacatctgactatgtaaaagaagcaaagcagttattaaaagtacaaaaagcatatcatcaacttgatcaatcatttatagacacttatattgatttattggaaacaagaagaacatattatgagggaccaggtgaaggtagcccatttggatggaaagatattaaagaatggtatgaaatgttaatgggacattgtacgtatttcccagaagaattacgtagtgtgaaatatgcctataatgctgatttatataatgcgctgaatgatttgaacaacttggttattacacgagatgagaatgagaagctagagtattatgaaaaattccaaattatcgagaatgtctttaaacaaaagaaaaagccgacgcttaaacaaattgcgaaggaaatcttggtgaatgaagaagacatcaaaggctatcgtgtcacaagtacaggtaaaccagaatttacaaacttgaaagtttatcacgatatcaaagatattacagcaagaaaagaaattatcgagaatgcagagctactcgatcaaatagctaaaatattaactatttaccagtcatcagaagatatacaagaagaattaacaaacctaaattcagaattgacacaagaagagattgaacaaatttcaaacttgaaaggttatacaggaactcataacctttcactaaaggcaataaatttaatattagacgaattgtggcatacgaacgataatcaaatagctattttcaatcgtttgaaacttgtacctaaaaaggtagatttaagccaacaaaaagaaattcctactactttagttgatgattttatactgtctccagtagtgaaacgttcatttatacaatctattaaagttattaacgctattattaaaaaatacggtttgccaaatgatattattattgaacttgcgagagaaaagaattctaaagatgcacaaaaaatgattaatgaaatgcagaagagaaatcgtcaaacgaatgaacgtattgaggaaattataagaacgacaggtaaagaaaatgctaaatatttaattgaaaaaattaagctgcacgatatgcaagaagggaaatgtttatactcgttagaagcaatccctctagaagatttacttaataatccattcaattacgaagtagaccatatcattccacgttctgtttctttcgataactctttcaataataaagtgttggtgaaacaagaagaaaatagtaaaaaaggtaatagaacgccatttcaatatttaagttcttcagattctaaaataagttatgagacattcaaaaagcatattttaaatcttgctaaaggcaaaggtagaatctctaagacgaaaaaagaatatttgttagaagaacgagatatcaatcgcttcagtgtccaaaaagattttattaaccgtaacttagtagatacacgctatgcgacaagagggttaatgaacttattaagatcttattttagagtgaataacttagatgtcaaagtgaaatcgattaatggcggattcacaagtttcttaagaaggaaatggaagttcaaaaaagaaagaaataagggctacaaacaccatgctgaagatgcactgattattgcgaacgctgattttattttcaaagaatggaaaaaactagataaagctaaaaaagtgatggaaaatcaaatgtttgaagaaaagcaagctgaaagtatgcctgaaattgagactgagcaagagtataaagaaatttttataacgcctcatcaaattaaacatattaaggattttaaagattataaatattcacatagagttgataaaaagccgaatagagagttaataaatgatacattatattctacgagaaaagatgacaagggtaatacattaatcgttaataacttaaatggtttatacgataaagataatgataaattgaaaaaattaattaataaatcacctgaaaaattattgatgtatcatcatgatccacaaacatatcaaaaattaaaattgatcatggaacaatatggcgatgagaaaaatccgctttataaatattatgaagaaacaggcaattacttaacaaaatatagtaaaaaagataacggaccagtcatcaaaaaaattaaatattatggtaacaagctaaatgcgcatttagatattacggatgattatccaaatagcagaaataaagtagtaaaactttcattaaaaccatatcgctttgatgtttatttagataatggggtatataaatttgtgacagttaaaaatttagatgttatcaaaaaagaaaactactatgaagttaattcaaagtgttatgaagaagcaaaaaaactgaagaaaattagtaatcaagcagaatttatcgcaagtttttacaataatgacttgattaagattaacggagaattatatagagtcataggtgtaaataatgatctacttaacagaattgaagtaaatatgatagacatcacatatagagaatatttagagaacatgaatgataaaagaccacctagaataattaaaacaatagcaagcaaaacacaatctattaaaaagtattctacagatattctaggcaatctttatgaagtgaagagtaaaaagcatcctcaaatcataaagaaaggatga

SEQ ID NO: 4 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 5 - DNA, 4107 bp, Streptococcus pyogenes
  source 1..4107: mol_type = genomic DNA
atggataagaaatactcaataggcttagatatcggcacaaatagcgtcggatgggcggtgatcactgatgattataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggcagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtagagtagatgctaaagcgattctttctgcacgattgagtaaatcaagacgattagaaaatctcattgctcagctccccggtgagaagaaaaatggattgtttgggaatctcattgctttgtcattgggattgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctactttactttcagatatcctaagagtaaatagtgaaataactaaggctcccctatcagcttcaatgattaagcgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggcgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgagggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggcacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagatattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgtgagcgtatgaaacgtattgaagaaggaataaaagaactaggaagtgatattctaaaggagtatcctgttgaaaacactcaattacaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatcttaatgccgtcgttggaactgctttgattaagaaatatccaaaacttgaatcggagtttgtctatggtgattataaagtttatgatgttcgtaaaatgattgctaagtctgagcaggaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttagaaaagatttaatcattaaactacctaaatatagtctttttgagttagaaaacggtcgtaaacggatgctggctagcgccggggaattgcaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccattactggtctttatgaaacacgcattgatttgagtcagctaggaggtgactga

SEQ ID NO: 6 - protein, 1368 aa, Streptococcus pyogenes
MDKKYSIGLDIGTNSVGWAVITDDYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRLNSEITKAPLSASMIKRYDEHHQDLTLLKALVRQQLPEKYKEIFFDQSKNGYAGYIDGGASQEEFYKFIKPILEKMDGTEELLAKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGLTIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD